(19) United States
(12) Patent Application Publication
(10) Pub. No.: US 2023/0039941 A1
(43) Pub. Date: Feb. 9, 2023

(54) ENZYME ENHANCED ROOT UPTAKE OF AGROCHEMICAL ACTIVE COMPOUND

(71) Applicant: BASF SE, Ludwigshafen (DE)

(72) Inventors: Wen Xu, Research Triangle Park, NC (US); Frederick Jaeger, Research Triangle Park, NC (US); Huazhang Huang, Durham, NC (US); Michael Warden, Research Triangle Park, NC (US); Adrienne Huston Davenport, San Diego, CA (US); Michael Liszka, San Diego, CA (US); Charles W. Finch, Research Triangle Park, NC (US)

(21) Appl. No.: 17/786,149

(22) PCT Filed: Dec. 21, 2020
(86) PCT No.: PCT/EP2020/087358

§ 371 Date: Jun. 16, 2022

Related U.S. Application Data
(60) Provisional application No. 62/952,606, filed on Dec. 23, 2019.

(30) Foreign Application Priority Data
Dec. 23, 2019 (EP) 20155077.9
Jun. 5, 2020 (EP) 20178504.5

(51) Int. Cl. A01N 63/27; C12N 9/18; A01N 43/90

(52) U.S. Cl. CPC A01N 63/27; C12N 9/18; A01N 43/90

(57) ABSTRACT

The presently claimed invention relates to a method for protecting plants or plant protecting material from attack or infestation by insects, fungi, arachnids, nematodes and unwanted weeds by treatment of root/shoot/seeds/soil by the use of active compounds in combina-tion with enzymes.

### FIELD OF THE INVENTION

The presently claimed invention relates to a method for protecting plants or plant protecting material from attack or infestation by insects, fungi, arachnids, nematodes and unwanted weeds by treatment of root/shoot/seeds/soil by the use of active compounds in combination with enzymes.

### BACKGROUND OF THE INVENTION

Agricultural chemicals including insecticides, fungicides, herbicides, miticides (or acaricides) and plant growth regulators have been used in the forms of, for example, emulsions, wettable powders, granules, dusts and flowables. In the properties of these agricultural chemical preparations, various attempts have been made to achieve the maximum efficacy of the agricultural chemical. However, it has been difficult to enhance the efficacies of agricultural chemicals through adjustments in formulations. It is further difficult to develop novel agricultural chemicals. Therefore, further enhancement of the efficacies of existing agricultural chemicals would highly contribute to the industry.

As substances capable of enhancing the efficacies of agricultural chemicals, surfactants comprising various nitrogen-containing compounds such as quaternary ammonium salts, betaines and amine oxides have been known. It is known that quaternized or further polyoxyethylenated long-chain amines, among the above-mentioned compounds, are effective for this purpose.

European Patent Publication. 0 184,288 discloses the use of an esterase such as lipase or a polysaccharase such as cellulase as an ingredient of a composition of a herbicide, insecticide, miticide or the like.

European Patent Publication 0 272 002 discloses the use of enzyme in combination with an agricultural chemical to improve the activity of the agricultural chemical.

U.S. Pat. No. 4,762,547 discloses the effect of enhancing the activities of agricultural chemicals in the presence of adjuvants. The effects of the surfactants comprising the nitrogen-containing compound and enzymes capable of enhancing the efficacies of agricultural chemicals are not always satisfactory.

WO 94/13784 relates to the use of an anti-fungal composition comprising the synergistic combination of a fungal cell wall degrading enzyme selected from chitinolytic enzymes, glucanolytic enzymes and cellulases and non-enzymatic fungicides selected from sterol synthesis inhibiting fungicides and thiol group inactivating fungicides.

Agrochemicals generally are taken up by the plants and they move with water in plants by the capillary action through xylem or phloem. Low water solubility of agrochemicals could lead to a low concentration in plants, therefore, lead to low efficacy against pests. Surfactants generally act on the cuticle of the seed or cell wall of roots, therefore, increase the chance of more molecules to enter the plants, but movement in plants depends on its systemicity. Non-systemic compounds generally have very low water solubility and high lipophilicity (Log P>4). Therefore, even though non-systemic compounds could enter plants with the aid of surfactants, their upward movement in the plants is limited.

Generally, the insecticides with low water solubility have low root uptake through seed treatment.

Thus, higher amounts of insecticide is required to achieve control of foliar insects.

Thus, it an object of the present invention to improve the uptake of the agricultural active ingredient through root uptake.

The further object of the present invention is to significantly reduce the use rate of -systemic agriculturally active compounds for seed treatment application.

### SUMMARY OF THE INVENTION

Surprising it was found that the use of enzymes helps to improve the penetration of the agricultural active through the root/seed cuticle.

Thus, in a first aspect, the presently claimed invention is directed to a method of protecting plants or plant propagation material from attack or infestation by insects, fungi, arachnids or nematodes, protection against weeds comprising application of:

at least one active compound, and

at least one enzyme selected from the group consisting of protease, peptidase, cellulase, hemicellulase, pectinase, chitinase, chitosanase, lipase, cutinase, esterase and xylanase, directly and/or indirectly to the plant and/or to plant propagation material by drenching the soil, by drip application onto the soil, by soil injection, by dipping, in-furrow application or by treatment of plant propagation material.

In a second aspect, the presently claimed invention is directed to the use of at least one active compound and at least one enzyme selected from the group consisting of protease, peptidase, cellulase, hemicellulase, pectinase, chitinase, chitosanase, lipase, cutinase, esterase and xylanase for protecting plants or plant propagation material from attack or infestation by insects, fungi, arachnids or nematodes or protection against weeds, wherein the at least one active compound and the at least one enzyme is applied directly and/or indirectly to the plant and/or to plant propagation material by drenching the soil, by drip application onto the soil, by soil injection, by dipping, in furrow application or by treatment of plant propagation material.

In a third aspect, the presently claimed invention is directed to a composition for protecting plants or plant propagation material from attack or infestation by insects, fungi, arachnids or nematodes or protection against weeds, comprising:

at least one active compound and

at least one enzyme selected from the group consisting of protease, peptidase, cellulase, hemi-cellulase, pectinase, chitinase, chitosanase, lipase, cutinase, esterase and xylanase.

In a fourth aspect, the presently claimed invention is directed to seeds comprising at least one active compound and at least one enzyme selected from the group consisting of protease, peptidase, cellulase, hemi-cellulase, pectinase, chitinase, chitosanase, lipase, cutinase, esterase and xylanase in an amount of from 1 ppb to 1 ppt per weight of seeds or plant propagation medium.

In a fifth aspect, the presently claimed invention is directed to kit of parts comprising, as separate components, at least one active compound for a combined use with at least one enzyme selected from the group consisting of protease, peptidase, cellulase, hemi-cellulase, pectinase, chitinase, chitosanase, lipase, cutinase, esterase and xylanase.

### DETAILED DESCRIPTION OF THE INVENTION

### Definitions

Before the present compositions and formulations of the invention are described, it is to be understood that this invention is not limited to compositions and formulations described, since such compositions and formulation may, of course, vary. It is also to be understood that the terminology used herein is not intended to be limiting, since the scope of the presently claimed invention will be limited only by the appended claims.

If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(A)”, “(B)” and “(C)” or “(a)”, “(b)”, “(c)”, “(d)”, “i”, “ii” etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the presently claimed invention. Thus, appearances of the phrases “in one embodiment” or “in an embodiment” in various places throughout this specification are not necessarily all referring to the same embodiment, but may do so. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art. For example, in the appended claims, any of the claimed embodiments can be used in any combination.

Furthermore, the ranges defined throughout the specification include the end values as well i.e. a range from 1 to 10 implies that both 1 and 10 are included in the range. For the avoidance of doubt, applicant shall be entitled to any equivalents according to applicable law.

As defined herein “agriculturally active compounds” are selected from pesticides, safeners and/or growth regulators. The pesticides are further defined as fungicides, insecticides, nematicides, herbicides.

Method

In an embodiment, the presently claimed invention is directed to a method of protecting plants or plant propagation material from attack or infestation by insects, fungi, arachnids or nematodes, protection against weeds comprising application of:

at least one active compound, and

at least one enzyme selected from the group consisting of protease, peptidase, cellulase, hemi-cellulase, pectinase, chitinase, chitosanase, lipase, cutinase, esterase and xylanase, directly and/or indirectly to the plant and/or to plant propagation material by drenching the soil, by drip application onto the soil, by soil injection, by dipping, in-furrow application or by treatment of plant propagation material.

In an embodiment of the present invention, the insecticides are selected from the group consisting of:

The following list M of pesticides, grouped according the Mode of Action Classification of the Insecticide Resistance Action Committee (IRAC), together with which the compounds of the invention can be used and with which potential synergistic effects might be produced, illustrates the possible combinations:

M.1 AChE inhibitors: aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb, triazamate; acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, malathion, mecarbam, methamido-phos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetam-phos, prothiofos, pyraclofos, pyridaphenthion, quinalphos, sulfotep, tebupirimfos, temeph-os, terbufos, tetrachlorvinphos, thiometon, triazophos, trichlorfon, vamidothion;

M.2. GABA-gated chloride channel antagonists: cyclodiene organochlorine compounds: endosulfan, chlordane; phenylpyrazoles: ethiprole, fipronil, flufiprole, pyrafluprole, pyriprole;

M.3 Sodium channel modulators: pyrethroids: acrinathrin, allethrin, d-cis-trans allethrin, d-trans allethrin, bifenthrin, kappa-bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, taufluvalinate, halfenprox, heptafluthrin, imiprothrin, meperfluthrin, metofluthrin, momfluorothrin, epsilon-momfluorothrin, permethrin, phenothrin, prallethrin, profluthrin, pyrethrin (pyrethrum), resmethrin, silafluofen, tefluthrin, kappa-tefluthrin, tetramethylfluthrin, tetramethrin, tralo-methrin, transfluthrin; sodium channel modulatorse.g.: DDT, methoxychlor;

M.4 nAChR agonists: neonicotinoids: acetamiprid, clothianidin, cycloxaprid, dinotefuran, imidacloprid, nitenpyram, thiacloprid, thiamethoxam; 4,5-Dihydro-N-nitro-1-(2-oxiranylmethyl)-1H-imidazol-2-amine, (2E-)-1-[(6-Chloropyridin-3-yl)methyl]-V-nitro-2-pentylidenehydrazinecarboximidamide; 1-[(6-Chloropyridin-3-yl)methyl]-7-methyl-8-nitro-5-propoxy-1,2,3,5,6,7-hexahydroimidazo[1,2-a]pyridine; nicotine; sulfoxaflor; flupyradifurone; triflumezopyrim, (3R)-3-(2-chlorothiazol-5-yl)-8-methyl-5-oxo-6-phenyl-2,3-dihydrothiazolo[3,2-a]pyrimidin-8-ium-7-olate, (3S)-3-(6-chloro-3-pyridyl)-8-methyl-5-oxo-6-phenyl-2,3-dihydrothiazolo[3,2-a]pyrimidin-8-ium-7-olate, (3S)-8-methyl-5-oxo-6-phenyl-3-pyrimidin-5-yl-2,3-dihydrothiazolo[3,2-a]pyrimidin-8-ium-7-olate, (3R)-3-(2-chlorothiazol-5-yl)-8-methyl-5-oxo-6-[3-(trifluoromethyl)phenyl]-2,3-dihydrothiazolo[3,2-a]pyrimidin-8-ium-7-olate; (3R)-3-(2-chlorothiazol-5-yl)-6-(3,5-dichlorophenyl)-8-methyl-5-oxo-2,3-dihydrothiazolo[3,2-a]pyrimidin-8-ium-7-olate, (3R)-3-(2-chlorothiazol-5-yl)-8-ethyl-5-oxo-6-phenyl-2,3-dihydrothiazolo[3,2-a]pyrimidin-8-ium-7-olate;

M.5 Nicotinic acetylcholine receptor allosteric activators: spinosad, spinetoram;

M.6 Chloride channel activators: abamectin, emamectin benzoate, ivermectin, lepimectin, milbemectin;

M.7 Juvenile hormone mimics e.g.: hydroprene, kino¬prene, methoprene; fenoxycarb, pyriproxyfen;

M.8 miscellaneous multi-site inhibitors: CH3Br, other alkyl halides, chloropicrin, sulfuryl fluoride, borax, tartar emetic;

M.9 Chordotonal organ TRPV channel modulators: pymetrozine; pyrifluquinazon;

M.10 Mite growth inhibitors: clofentezine, hexythiazox, diflovidazin, etoxazole;

M.11 Microbial disruptors of insect midgut membranes: Bacillus thuringiensis, Bacillus sphaericus, and insecticidal proteins they produce e.g.: Bacillus thuringiensis subsp. israelensis, Bacillus sphaericus, Bacillus thuringiensis subsp. aizawai, Bacillus thuringiensis subsp. kurstaki, Bacillus thuringiensis subsp. tenebrionis, Bt crop proteins: Cry1Ab, Cry1Ac, Cry1Fa, Cry2Ab, mCry3A, Cry3Ab, Cry3Bb, Cry34/35Ab1;

M.12 Inhibitors of mitochondrial ATP synthase: diafenthiuron, organotin miticidese.g.: azocyclotin, cyhexatin, fenbutatin oxide, propargite, tetradifon;

M.13 Uncouplers of oxidative phosphorylation via disruption of the proton gradient: chlorfenapyr, DNOC, sulfluramid;

M.14 nAChR channel blockers: nereistoxin analogues bensultap, cartap hydrochloride, thiocyclam, thiosultap-sodium;

M.15 Inhibitors of the chitin biosynthesis type e.g.: bistrifluron, chlorfluazuron, difluben-zuron, flucycloxuron, flufenoxuron, hexaflumuron, lufenuron, novaluron, noviflumuron, teflubenzuron, triflumuron;

M.16 Inhibitors of the chitin biosynthesis type 1: buprofezin;

M.17 Moulting disruptors: Dipteran, cyromazine;

M.18 Ecdyson receptor agonistse.g.: methoxyfenozide, tebufenozide, halofenozide, fufe-nozide, chromafenozide;

M.19 Octopamin receptor agonists: amitraz;

M.20 Mitochondrial complex III electron transport inhibitors: hydramethylnon, acequi-nocyl, fluacrypyrim; bifenazate;

M.21 METI acaricides and insecticides e.g.: fenazaquin, fenpyroximate, pyrimidifen, pyridaben, tebufenpyrad, tolfenpyrad, rotenone;

M.22 Voltage-dependent sodium channel blockers: indoxacarb, metaflumizone, 2-[2-(4-cyanophenyl)-1-[3-(trifluoromethyl)phenyl]¬ethylidene]-N-[4-(difluoromethoxy)phenyl]-hydrazinecarboxamide, N-(3-chloro-2-methyl¬phenyl)-2-[(4-chlorophenyl)[4-[methyl(methylsulfonyl)amino]phenyl]¬methylene]-hydrazinecarboxamide;

M.23 Inhibitors of the of acetyl CoA carboxylase e.g.: spirodiclofen, spiromesifen, spirotetramat; spiropidion;

M.24 Mitochondrial complex IV electron transport inhibitors: e.g. aluminium phosphide, calcium phosphide, zinc phosphide, cyanide;

M.25 Mitochondrial complex II electron transport inhibitors e.g.: cyenopyrafen, cyflumetofen;

M.28 Ryanodine receptor-modulators: flubendiamide, chlorantraniliprole, cyantraniliprole, tetraniliprole, (R)-3-chloro-N1-{2-methyl-4-[1,2,2,2-tetrafluoro-1-(trifluoromethyl)ethyl]phenyl}-N2-(1-methyl-2-methylsulfonylethyl)phthalamid, (S)-3-chloro-N1-{2-methyl-4-[1,2,2,2-tetrafluoro-1-(trifluoromethyl)ethyl]phenyl}-N2-(1-methyl-2-methylsulfonylethyl)phthalamide, cyclaniliprole, methyl-2-[3,5-dibromo-2-({[3-bromo-1-(3-chlorpyridin-2-yl)-1H-pyrazol-5-yl]carbonyl}amino)benzoyl]-1,2-dimethylhydrazine-carboxylate; N-[2-(5-amino-1,3,4-thiadiazol-2-yl)-4-chloro-6-methylphenyl]-3-bromo-1-(3-chloro-2-pyridinyl)-1H-pyrazole-5-carboxamide; 3-chloro-1-(3-chloro-2-pyridinyl)-N-[2,4-dichloro-6-[[(1-cyano-1-methylethyl)amino]carbonyl]phenyl]-1H-pyrazole-5-carboxamide; tetrachlorantraniliprole; N-[4-chloro-2-[[(1,1-dimethylethyl)amino]carbonyl]-6-methyl¬phenyl]-1-(3-chloro-2-pyridinyl)-3-(fluoromethoxy)-1H-pyrazole-5-carboxamide; cyhalodiamide;

M.29: Chordotonal organ Modulators: flonicamid;

M.UN. Unknown mode of action: afidopyropen, afoxolaner, azadirachtin, amidoflumet, benzoximate, broflanilide, bromopropylate, chinomethionat, cryolite, dicloromezotiaz, dicofol, dimpropyridaz, flufenerim, flometoquin, fluensulfone, fluhexafon, fluopyram, fluralaner, metaldehyde, metoxadiazone, piperonyl butoxide, pyflubumide, pyridalyl, tioxazafen, 11-(4-chloro-2,6-dimethylphenyl)-12-hydroxy-1,4-dioxa-9-azadispiro[4.2.4.2]-tetradec-11-en-10-one, 3-(4′-fluoro-2,4-dimethylbiphenyl-3-yl)-4-hydroxy-8-oxa-1-azaspiro[4.5]dec-3-en-2-one, 1-[2-fluoro-4-methyl-5-[(2,2,2-trifluoroethyl)sulfinyl]phenyl]-3-(trifluoromethyl)-1H-1,2,4-triazole-5-amine, actives on basis of Bacillus firmus (Votivo, I-1582); flupyrimin; fluazaindolizine; 4-[5-(3,5-dichlorophenyl)-5-(trifluoromethyl)-4H-isoxazol-3-yl]-2-methyl-N-(1-oxothietan-3-yl)benzamide; fluxametamide; 5-[3-[2,6-dichloro-4-(3,3-dichloroallyloxy)phenoxy]propoxy]-1H-pyrazole; 4-cyano-N-[2-cyano-5-[[2,6-dibromo-4-[1,2,2,3,3,3-hexafluoro-1-(trifluoromethyl)-propyl]phenyl]carbamoyl]phenyl]-2-methyl-benzamide; 4-cyano-3-[(4-cyano-2-methyl-benzoyl)amino]-N-[2,6-dichloro-4-[1,2,2,3,3,3-hexafluoro-1-(trifluoromethyl)propyl]phenyl]-2-fluoro-benzamide; N-[5-[[2-chloro-6-cyano-4-[1,2,2,3,3,3-hexafluoro-1-(trifluoromethyl)propyl]phenyl]carbamoyl]-2-cyano-phenyl]-4-cyano-2-methyl-benzamide; N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide; N-[5-[[2-bromo-6-chloro-4-[1,2,2,3,3,3-hexafluoro-1-(trifluoromethyl)-propyl]phenyl]carbamoyl]-2-cyano-phenyl]-4-cyano-2-methyl-benzamide; 4-cyano-N-[2-cyano-5-[[2,6-dichloro-4-[1,2,2,3,3,3-hexafluoro-1-(trifluoromethyl)-propyl]phenyl]carbamoyl]phenyl]-2-methyl-benzamide; 4-cyano-N-[2-cyano-5-[[2,6-dichloro-4-[1,2,2,2-tetrafluoro-1-(trifluoromethyl)ethyl]phenyl]carbamoyl]¬phenyl]-2-methyl-benzamide; N-[5-[[2-bromo-6-chloro-4-[1,2,2,2-tetrafluoro-1-(trifluoromethyl)ethyl]phenyl]carba¬moyl]-2-cyano-phenyl]-4-cyano-2-methyl-benzamide; 2-(1,3-dioxan-2-yl)-6-[2-(3-pyridinyl)-5-thiazolyl]-pyridine; 2-[6-[2-(5-fluoro-3-pyridinyl)-5-thiazolyl]-2-pyridinyl]-pyrimidine; 2-[6-[2-(3-pyridinyl)-5-thiazolyl]-2-pyridinyl]-pyrimidine; N-methylsul¬fonyl-6-[2-(3-pyridyl)thiazol-5-yl]pyridine-2-carboxamide; N-methylsulfonyl-6-[2-(3-pyridyl)thiazol-5-yl]pyridine-2-carboxamide; 1-[(6-chloro-3-pyridinyl)methyl]-1,2,3,5,6,7-hexahydro-5-methoxy-7-methyl-8-nitro-imidazo[1,2-a]pyridine; 1-[(6-chloropyridin-3-yl)methyl]-7-methyl-8-nitro-1,2,3,5,6,7-hexahydroimidazo[1,2-a]pyridin-5-ol; N-(1-methylethyl)-2-(3-pyridinyl)-2H-indazole-4-carboxamide; N-cyclopropyl-2-(3-pyridinyl)-2H-indazole-4-carboxamide; N-cyclohexyl-2-(3-pyridinyl)-2H-indazole-4-carboxamide; 2-(3-pyridinyl)-N-(2,2,2-trifluoroethyl)-2H-indazole-4-carboxamide; 2-(3-pyridinyl)-N-[(tetrahydro-2-furanyl)methyl]-2H-indazole-5-carboxamide; methyl 2-[[2-(3-pyridinyl)-2H-indazol-5-yl]carbonyl]hydrazinecarboxylate; N-[(2,2-difluorocyclopropyl)methyl]-2-(3-pyridinyl)-2H-indazole-5-carboxamide; N-(2,2-difluoropropyl)-2-(3-pyridinyl)-2H-indazole-5-carboxamide; 2-(3-pyridinyl)-N-(2-pyrimidinylmethyl)-2H-indazole-5-carboxamide; N-[(5-methyl-2-pyrazinyl)methyl]-2-(3-pyridinyl)-2H-indazole-5-carboxamide, tyclopyrazoflor; sarolaner, lotilaner; N-[4-chloro-3-[[(phenyl methyl)amino]carbonyl]phenyl]-1-methyl-3-(1,1,2,2,2-pentafluoroethyl)-4-(trifluoromethyl)-1H-pyrazole-5-carboxamide; 2-(3-ethylsulfonyl-2-pyridyl)-3-methyl-6-(tri¬fluoromethyl)imidazo[4,5-b]pyridine, 2-[3-ethylsulfonyl-5-(trifluoromethyl)-2-pyridyl]-3-methyl-6-(trifluoromethyl)imidazo[4,5-b]pyridine; Isocycloseram; N-[4-chloro-3-(cyclopropylcarbamo¬yl)phenyl]-2-methyl-5-(1,1,2,2,2-pentafluoroethyl)-4-(trifluoromethyl)pyrazole-3-carboxamide, N-[4-chloro-3-[(1-cyanocyclopropyl)carbamoyl]phenyl]-2-methyl-5-(1,1,2,2,2-pentafluoroethyl)-4-(trifluoromethyl)pyrazole-3-carboxamide; acynonapyr; benzpyrimoxan; tigolaner; oxazosulfyl; [(2S,3R,4R,5S,6S)-3,5-dimethoxy-6-methyl-4-propoxy-tetrahydropyran-2-yl] N-[4-[1-[4-(trifluoromethoxy)phenyl]-1,2,4-triazol-3-yl]phenyl]carbamate; [(2S,3R,4R,5S,6S)-3,4,5-trimethoxy-6-methyl-tetrahydropyran-2-yl] N-[4-[1-[4-(trifluoromethoxy)phenyl]-1,2,4-triazol-3-yl]phenyl]carbamate; [(2S,3R,4R,5S,6S)-3,5-dimethoxy-6-methyl-4-propoxy-tetrahydropyran-2-yl] N-[4-[1-[4-(1,1,2,2,2-pentafluoroethoxy)phenyl]-1,2,4-triazol-3-yl]phenyl]carbamate; [(2S,3R,4R,5S,6S)-3,4,5-trimethoxy-6-methyl-tetrahydropyran-2-yl] N-[4-[1-[4-(1,1,2,2,2-pentafluoroethoxy)phenyl]-1,2,4-triazol-3-yl]phenyl]carbamate; (2Z)-3-(2-isopropylphenyl)-2-[(E)-[4-[1-[4-(trifluoromethoxy)phenyl]-1,2,4-triazol-3-yl]phenyl]methylenehydrazono]thiazolidin-4-one, (2Z)-3-(2-isopro¬pyl¬phenyl)-2-[(E)-[4-[1-[4-(1,1,2,2,2-pentafluoroethoxy)phenyl]-1,2,4-triazol-3-yl]phenyl]methylenehydrazono]thiazolidin-4-one; 2-(6-chloro-3-ethylsulfonyl-imidazo[1,2-a]pyridin-2-yl)-3-methyl-6-(trifluoromethyl)imidazo[4,5-b]pyridine, 2-(6-bromo-3-ethylsulfonyl-imidazo[1,2-a]pyridin-2-yl)-3-methyl-6-(trifluoromethyl)imidazo[4,5-b]pyridine, 2-(3-ethylsulfonyl-6-iodo-imidazo[1,2-a]pyridin-2-yl)-3-methyl-6-(trifluoromethyl)imidazo[4,5-b]pyridine, 2-[3-ethylsulfonyl-6-(trifluoromethyl)imidazo[1,2-a]pyridin-2-yl]-3-methyl-6-(trifluoromethyl)imidazo[4,5-b]pyridine, 2-(7-chloro-3-ethylsulfonyl-imidazo[1,2-a]pyridin-2-yl)-3-methyl-6-(trifluoromethyl)imidazo[4,5-b]pyridine, 2-(3-ethylsulfonyl-7-iodo-imidazo[1,2-a]pyridin-2-yl)-3-methyl-6-(trifluoromethyl)imidazo[4,5-b]pyridine, 3-ethylsulfonyl-6-iodo-2-[3-methyl-6-(trifluoromethyl)imidazo[4,5-b]pyridin-2-yl]imidazo[1,2-a]pyridine-8-carbonitrile, 2-[3-ethylsulfonyl-8-fluoro-6-(trifluoromethyl)imidazo[1,2-a]pyridin-2-yl]-3-methyl-6-(trifluoromethyl)imidazo[4,5-b]pyridine, 2-[3-ethylsulfonyl-7-(trifluoromethyl)imidazo[1,2-a]pyridin-2-yl]-3-methyl-6-(trifluoromethylsulfinyl)imidazo[4,5-b]pyridine, 2-[3-ethylsulfonyl-7-(trifluoromethyl)imidazo[1,2-a]pyridin-2-yl]-3-methyl-6-(trifluoromethyl)imidazo[4,5-c]pyridine, 2-(6-bromo-3-ethylsulfonyl-imidazo[1,2-a]pyridin-2-yl)-6-(trifluoromethyl)pyrazolo[4,3-c]pyridine.

The commercially available compounds M listed above may be found in The Pesticide Manual, 18th Edition, C. MacBean, British Crop Protection Council (2018), or http://bcpcdata.com/pesticide-manual.html, http://www.alanwood.net/pesticides.

The active compounds described by IUPAC nomenclature are known from CN103814937; WO2013/003977, WO2007/101369, WO2018/177970, CN10171577, CN102126994, WO2007/101540, WO2007/043677, WO2011/085575, WO2008/134969, WO2012/034403, WO2006/089633, WO2008/067911, WO2006/043635, WO2009/124707, WO2013/050317, WO2010/060379, WO2010/127926, WO2010/006713, WO2012/000896, WO2007/101369, WO2012/143317, WO2015/038503, EP2910126, WO2015/059039, WO2015/190316, WO2012/126766, WO2009/102736, WO2013/116053, WO2018/052136.

In an embodiment of the present invention, the fungicides are selected from the group consisting of the following list:

### A) Respiration Inhibitors

Inhibitors of complex III at Qo site: azoxystrobin, coumethoxystrobin, coumoxystrobin, dimoxystrobin, enestroburin, fenaminstrobin, fenoxystrobin/flufenoxystrobin, fluoxastrobin, kresoxim-methyl, mandestrobin, metominostrobin, orysastrobin, picoxystrobin, pyraclostrobin, pyrametostrobin, pyraoxystrobin, trifloxystrobin, 2 (2-(3-(2,6-di¬chlorophenyl)-1-methyl-allylideneaminooxymethyl)-phenyl)-2 methoxyimino-N methyl-acetamide, pyri-bencarb, triclopyricarb/chlorodincarb, famoxadone, fenamidone, methyl-N-[2-[(1,4-dimethyl-5 phenyl-pyrazol-3-yl)oxylmethyl]phenyl]-N-methoxycarbamate, metyltetrapole, (Z,2E) 5 [1-(2,4-dichloro¬phenyl)pyrazol-3-yl]-oxy-2-methoxyimino-N,3-dimethyl-pent-3-en¬amide, (Z,2E) 5 [1 (4-chlorophenylpyrazol-3-yl]oxy-2-methoxyimino-N,3-dimethyl-pent-3-enamide, pyriminostrobin, bifujunzhi, 2-(ortho-((2,5-dimethylphenyl-oxy-methylen)phenyl)-3-methoxy-acrylic acid methylester; inhibitors of complex III at Qi site: cyazofamid, amisulbrom, [(6S,7R,8R) 8 benzyl-3-[(3-hydroxy-4-methoxy-pyridine-2-carbonyl)amino]-6-methyl-4,9-di¬oxo-1,5-dioxonan-7-yl]2-methylpropanoate, fenpicoxamid, florylpicoxamid;
inhibitors of complex II: benodanil, benzovindiflupyr, bixafen, boscalid, carboxin, fenfuram, fluopyram, flutolanil, fluxapyroxad, furametpyr, isofetamid, isopyrazam, mepronil, oxycarboxin, penflufen, penthiopyrad), pydiflumetofen, pyraziflumid, sedaxane, tecloftalam, thifluzamide, inpyrfluxam, pyrapropoyne, fluindapyr, N-[2-[2-chloro-4-(trifluoro-methyl)phenoxy]phenyl]-3-(difluoromethyl)-5-fluoro-1-methyl-pyrazole-4-carboxamide, methyl (E)-2-[2-[(5-cyano-2-methyl-phenoxy)methyl]phenyl]-3-methoxyprop-2 enoate, isoflucypram, 2-(difluoromethyl)-N-(1,1,3-trimethyl-indan-4 yl)pyridine-3-carboxamide, 2-(difluoromethyl)-N-[(3R)-1,1,3-trimethylindan-4-yl]pyridine-3-carboxamide, 2-(difluoromethyl)-N-(3-ethyl-1,1-dimethyl-indan-4-yl)pyridine-3-carboxamide, 2-(difluoromethyl)-N-[(3R)-3-ethyl-1,1-dimethyl-indan-4-yl]pyridine-3-carboxamide, 2-(difluoromethyl)-N-(1,1-dimethyl-3-propyl-indan-4-yl)pyridine-3-carboxamide, 2-(difluoromethyl)-N-[(3R)-1,1-dimethyl-3-propyl-indan-4-yl]pyridine-3-carboxamide, 2-(difluoromethyl)-N-(3-isobutyl-1,1-dimethyl-indan-4-yl)pyridine-3-carboxamide, 2-(difluoromethyl)-N-[(3R)-3-isobutyl-1,1-dimethyl-indan-4 yl]pyridine-3-carboxamide;
other respiration inhibitors: diflumetorim; nitrophenyl derivates: binapacryl, dinobuton, dinocap, fluazinam, meptyldinocap, ferimzone; organometal compounds: fentin salts, e.g. fentin-acetate, fentin chloride, fentin hydroxide; ametoctradin; silthiofam;

### B) Sterol Biosynthesis Inhibitors (SBI Fungicides)

C14 demethylase inhibitors: triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 2 (2,4-di¬fluorophenyl)-1,1-difluoro-3-(tetrazol-1-yl)-1-[5-[4-(2,2,2-trifluoroethoxy)phenyl]-2 pyridyl]propan-2-ol, 2-(2,4-difluorophenyl)-1,1-difluoro-3-(tetrazol-1-yl)-1 [5 [4 (trifluoromethoxy)phenyl]-2-pyridyl]propan-2-ol, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile, ipfentrifluconazole, mefentrifluconazole, 2-(chloromethyl)-2-methyl-5-(p-tolylmethyl)-1 (1,2,4-triazol-1 ylmethyl)cyclopentanol; imidazoles: imazalil, pefurazoate, prochloraz, triflumizol; pyrimidines, pyridines, piperazines: fenarimol, pyrifenox, triforine, [3-(4-chloro-2-fluoro-phenyl)-5-(2,4-di¬fluoro¬phenyl)isoxazol-4-yl]-(3-pyridyl)methanol;
Delta14-reductase inhibitors: aldimorph, dodemorph, dodemorph-acetate, fenpropimorph, tridemorph, fenpropidin, piperalin, spiroxamine;
Inhibitors of 3-keto reductase: fenhexamid;
Other Sterol biosynthesis inhibitors: chlorphenomizole;

### C) Nucleic Acid Synthesis Inhibitors

phenylamides or acyl amino acid fungicides: benalaxyl, benalaxyl-M, kiralaxyl, metalaxyl, metalaxyl-M, ofurace, oxadixyl;
other nucleic acid synthesis inhibitors: hymexazole, octhilinone, oxolinic acid, bupirimate, 5-fluorocytosine, 5-fluoro-2-(p-tolylmethoxy)pyrimidin-4 amine, 5-fluoro-2-(4-fluorophenylmethoxy)pyrimidin-4 amine, 5-fluoro-2 (4 chlorophenylmethoxy)pyrimidin-4 amine;

### D) Inhibitors of Cell Division and Cytoskeleton

tubulin inhibitors: benomyl, carbendazim, fuberidazole, thiabendazole, thiophanate-methyl, pyridachlometyl, N ethyl-2-[(3-ethynyl-8-methyl-6-quinolyl)oxy]butanamide, N-ethyl-2-[(3-ethynyl-8 methyl-6 quinolyl)oxy]-2-methylsulfanyl-acetamide, 2-[(3-ethynyl-8-methyl-6-quinolyl)oxy]-N(2-fluoroethyl)butanamide, 2-[(3-ethynyl-8-methyl-6-quinolyl)oxy]-N-(2-fluoroethyl)-2-methoxy-acetamide, 2-[(3-ethynyl-8-methyl-6-quinolyl)oxy]-N-propyl-butanamide, 2-[(3-ethynyl-8-methyl-6-quinolyl)oxy]-2-methoxy-N-propyl-acetamide, 2-[(3-ethynyl-8-methyl-6-quinolyl)oxy]-2-methylsulfanyl-N-propyl-acetamide, 2 [(3 ethynyl-8-methyl-6-quinolyl)oxy]-N-(2-fluoroethyl)-2-methyl-sulfanylacetamide, 4-(2-bromo-4-fluoro-phenyl)-N-(2-chloro-6-fluoro-phenyl)-2,5-dimethylpyrazol-3 amine;
other cell division inhibitors: diethofencarb, ethaboxam, pencycuron, fluopicolide, zoxamide, metrafenone, pyriofenone, phenamacril;

### E) Inhibitors of Amino Acid and Protein Synthesis

methionine synthesis inhibitors: cyprodinil, mepanipyrim, pyrimethanil;
protein synthesis inhibitors: blasticidin-S, kasugamycin, kasugamycin hydrochloride-hydrate, mildiomycin, streptomycin, oxytetracyclin;

### F) Signal Transduction Inhibitors

MAP/histidine kinase inhibitors: fluoroimid, iprodione, procymidone, vinclozolin, fludioxonil; G protein inhibitors: quinoxyfen;

### G) Lipid and Membrane Synthesis Inhibitors

Phospholipid biosynthesis inhibitors: edifenphos, iprobenfos, pyrazophos, isoprothiolane; lipid peroxidation: dicloran, quintozene, tecnazene, tolclofos-methyl, biphenyl, chloroneb, etridiazole, zinc thiazole;
phospholipid biosynthesis and cell wall deposition: dimethomorph, flumorph, mandipropamid, pyrimorph, benthiavalicarb, iprovalicarb, valifenalate;
compounds affecting cell membrane permeability and fatty acids: propamocarb;
inhibitors of oxysterol binding protein: oxathiapiprolin, fluoxapiprolin, 4-[1-[2-[3-(difluoromethyl)-5-methyl-pyrazol-1-yl]acetyl]-4-piperidyl]-N-tetralin-1-yl-pyridine-2-carboxamide, 4-[1-[2-[3,5-bis(difluoromethyl)pyrazol-1-yl]acetyl]-4-piperidyl]-N-tetralin-1-yl-pyridine-2-carboxamide, 4-[1-[2-[3-(difluoromethyl)-5-(trifluoromethyl)pyrazol-1-yl]acetyl]-4-piperidyl]-N-tetralin-1-yl-pyridine-2-carboxamide, 4-[1-[2-[5-cyclopropyl-3-(difluoromethyl)pyrazol-1-yl]acetyl]-4-piperidyl]-N-tetralin-1-yl-pyridine-2-carboxamide, 4-[1-[2-[5-methyl-3-(trifluoromethyl)pyrazol-1-yl]acetyl]-4-piperidyl]-N-tetralin-1-yl-pyridine-2-carboxamide, 4-[1-[2-[5-(difluoromethyl)-3-(trifluoro¬methyl)pyrazol-1-yl]acetyl]-4-piperidyl]-N-tetralin-1-yl-pyridine-2-carboxamide, 4 [1 [2-[3,5-bis(trifluoromethyl)pyrazol-1-yl]acetyl]-4-piperidyl]-N-tetralin-1-yl-pyridine-2-car-boxamide, (4-[1-[2-[5-cyclopropyl-3-(trifluoromethyl)pyrazol-1-yl]acetyl]-4-piperidyl]-N-tetralin-1-yl-pyridine-2-carboxamide;
   H) Inhibitors with Multi Site Action
inorganic active substances: Bordeaux mixture, copper, copper acetate, copper hydroxide, copper oxychloride, basic copper sulfate, sulfur;
thio- and dithiocarbamates: ferbam, mancozeb, maneb, metam, metiram, propineb, thiram, zineb, ziram;
organochlorine compounds: anilazine, chlorothalonil, captafol, captan, folpet, dichlofluanid, dichlorophen, hexachlorobenzene, pentachlorphenole and its salts, phthalide, tolylfluanid;
guanidines and others: guanidine, dodine, dodine free base, guazatine, guazatine-acetate, iminoctadine, iminoctadine-triacetate, iminoctadine-tris(albesilate), dithianon, 2,6-di¬methyl-1H,5H-[1,4]dithiino[2,3-c:5,6-c′]dipyrrole-1,3,5,7(2H,6H)-tetraone;

### I) Cell Wall Synthesis Inhibitors

inhibitors of glucan synthesis: validamycin, polyoxin B;
melanin synthesis inhibitors: pyroquilon, tricyclazole, carpropamid, dicyclomet, fenoxanil;
   J) Plant defence inducers
acibenzolar-S-methyl, probenazole, isotianil, tiadinil, prohexadione-calcium; phosphonates: fosetyl, fosetyl-aluminum, phosphorous acid and its salts, calcium phosphonate, potassium phosphonate, potassium or sodium bicarbonate, 4 cyclopropyl-N-(2,4-dimethoxyphenyl)thiadiazole-5-carboxamide;

### K) Unknown Mode of Action

bronopol, chinomethionat, cyflufenamid, cymoxanil, dazomet, debacarb, diclocymet, diclomezine, difenzoquat, difenzoquat-methylsulfate, diphenylamin, fenitropan, fenpyrazamine, flumetover, flusulfamide, flutianil, harpin, methasulfocarb, nitrapyrin, nitrothal-isopropyl, tolprocarb, oxin-copper, proquinazid, tebufloquin, tecloftalam, triazoxide, N′-(4-(4-chloro-3-trifluoromethyl-phenoxy)-2,5-dimethyl-phenyl)-N-ethyl-N methyl formamidine, N′ (4-(4-fluoro-3-trifluoromethyl-phenoxy)-2,5-dimethyl-phenyl)-N-ethyl-N-methyl formamidine, N′-[4-[[3-[(4-chlorophenyl)methyl]-1,2,4-thiadiazol-5-yl]oxy]-2,5-dimethyl-phenyl]-N-ethyl-N-methyl-formamidine, N′-(5-bromo-6-indan-2-yloxy-2-methyl-3-pyridyl)-N-ethyl-N-methyl-formamidine, N′-[5-bromo-6-[1-(3,5-difluorophenyl)ethoxy]-2-methyl-3-pyridyl]-N-ethyl-N-methyl-formamidine, N′-[5-bromo-6-(4-isopropylcyclohexoxy)-2-methyl-3-pyridyl]-N-ethyl-N-methyl-formamidine, N′ [5 bromo-2-methyl-6-(1-phenylethoxy)-3-pyridyl]-N-ethyl-N-methyl-formamidine, N′-(2-methyl-5-trifluoromethyl-4-(3-trimethylsilanyl-prop¬oxy)-phenyl)-N-ethyl-N-methyl formamidine, N′-(5-difluoromethyl-2 methyl-4-(3-trimethylsilanyl-propoxy)-phenyl)-N-ethyl-N-methyl formamidine, 2-(4-chloro-phenyl)-N-[4-(3,4-dimethoxy-phenyl)-isoxazol-5 yl]-2-prop-2-ynyloxy-acetamide, 3 [5-(4-chloro-phenyl)-2,3-dimethyl-isoxazolidin-3-yl]-pyridine (pyrisoxazole) 3-[5-(4-methylphenyl)-2,3-dimethyl-isoxazolidin-3 yl]-pyridine, 5-chloro-1 (4,6-di¬methoxy-pyrimidin-2-yl)-2-methyl-1H-benzoimidazole, ethyl (Z) 3 amino-2-cyano-3-phenyl-prop-2-enoate (K.1.40), picarbutrazox, pentyl N-[6-[[(Z)[(1-methyltetrazol-5-yl)-phenyl-methylene]amino]oxymethyl]-2-pyridyl]carbamate, but-3-ynyl N-[6-[[(Z)-[(1-methyltetrazol-5-yl)-phenyl-methylene]amino]oxymethyl]-2-pyridyl]carbamate, ipflufenoquin, quinofumelin, benziothiazolinone, bromothalonil, 2-(6-benzyl-2-pyridyl)quinazoline, 2-[6-(3-fluoro-4 methoxy-phenyl)-5-methyl-2-pyridyl]-quinazoline, dichlobentiazox, N′-(2,5-dimethyl-4-phenoxy-phenyl)-N-ethyl-N-methyl-formamidine, pyrifenamine, fluopimomide, N′-[5-bromo-2-methyl-6-(1-methyl-2 propoxy-ethoxy)-3-pyridyl]-N-ethyl-N-methyl-formamidine.

The fungicides described by common names, their preparation and their activity e.g. against harmful fungi is known (cf.: http://www.alanwood.net/pesticides/); these sub-stances are commercially available.

The fungicides referred above, their preparation and their activity e.g. against harmful fungi is known (cf.: http://www.alanwood.net/pesticides/); these sub-stances are commercially available. The compounds described by IUPAC nomenclature, their preparation and their pesticidal activity are also known (cf. Can. J. Plant Sci. 48(6), 587-94, 1968; EP 141 317; EP 152 031; EP 226 917; EP 243 970; EP 256 503; EP 428 941; EP 532 022; EP 1 028 125; EP 1 035 122; EP 1 201 648; EP 1 122 244, JP 2002316902; DE 19650197; DE 10021412; DE 102005009458; U.S. Pat. Nos. 3,296,272; 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 10/139271, WO 11/028657, WO 12/168188, WO 07/006670, WO 11/77514; WO 13/047749, WO 10/069882, WO 13/047441, WO 03/16303, WO 09/90181, WO 13/007767, WO 13/010862, WO 13/127704, WO 13/024009, WO 13/24010, WO 13/047441, WO 13/162072, WO 13/092224, WO 11/135833, CN 1907024, CN 1456054, CN 103387541, CN 1309897, WO 12/84812, CN 1907024, WO 09094442, WO 14/60177, WO 13/116251, WO 08/013622, WO 15/65922, WO 94/01546, EP 2865265, WO 07/129454, WO 12/165511, WO 11/081174, WO 13/47441). Some compounds are identified by their CAS Registry Number.

In an embodiment of the present invention the herbicides are selected from

Acetamides: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor;

Amino acid derivatives: bilanafos, glyphosate, glufosinate, sulfosate;

Aryloxyphenoxypropionates: clodinafop, cyhalofop-butyl, fenoxaprop, fluazifop, haloxyfop, metamifop, propaquizafop, quizalofop, quizalofop-P-tefuryl;

Bipyridyls: diquat, paraquat;

(thio)carbamates: asulam, butylate, carbetamide, desmedipham, dimepiperate, eptam (EPTC), esprocarb, molinate, orbencarb, phenmedipham, prosulfocarb, pyributicarb, thiobencarb, triallate;

Cyclohexanediones: butroxydim, clethodim, cycloxydim, profoxydim, sethoxydim, tepraloxydim, tralkoxydim;

Dinitroanilines: benfluralin, ethalfluralin, oryzalin, pendimethalin, prodiamine, trifluralin;

Diphenyl ethers: acifluorfen, aclonifen, bifenox, diclofop, ethoxyfen, fomesafen, lactofen, oxyfluorfen;

Hydroxybenzonitriles: bomoxynil, dichlobenil, ioxynil;

Imidazolinones: imazamethabenz, imazamox, imazapic, imazapyr, imazaquin, imazethapyr;

Phenoxy acetic acids: clomeprop, 2,4-dichlorophenoxyacetic acid (2,4-D), 2,4-DB, dichlorprop, MCPA, MCPA-thioethyl, MCPB, Mecoprop;

Pyrazines: chloridazon, flufenpyrethyl, fluthiacet, norflurazon, pyridate;

Pyridines: aminopyralid, clopyralid, diflufenican, dithiopyr, fluridone, fluroxypyr, picloram, picolinafen, thiazopyr, triclopyr (2-[(3,5,6-trichloro-2-pyridinyl)oxy] acetic acid, butoxyethyl ester);

Sulfonyl ureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metazosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propyl-imidazo[1,2-b]pyridazin-3-yl)sulfonyl)-3-(4,6-dimethoxy-pyrimidin-2-yl)urea;

Triazines: ametryn, atrazine, cyanazine, dimethametryn, ethiozin, hexazinone, metamitron, metribuzin, prometryn, simazine, terbuthylazine, terbutryn, triaziflam; Ureas: chlorotoluron, daimuron, diuron, fluometuron, isoproturon, linuron, methabenzthiazuron, tebuthiuron;

Other acetolactate synthase inhibitors: bispyribac-sodium, cloransulam-methyl, diclosulam, florasulam, flucarbazone, flumetsulam, metosulam, ortho-sulfamuron, penoxsulam, propoxy carbazone, pyribambenz-propyl, pyribenzoxim, pyriftalid, pyriminobac-methyl, pyrimisulfan, pyrithiobac, pyroxasulfone, pyroxsulam;

Others: amicarbazone, aminotriazole, anilofos, beflubutamid, benazolin, bencarbazone, benfluresate, benzofenap, bentazone, benzobicyclon, bicyclopyrone, bromacil, bromobutide, butafenacil, butamifos, cafenstrole, carfentrazone, cinidon-ethyl, chlorthal, cinmethylin, clomazone, cumyluron, cyprosulfamide, dicamba, difenzoquat, diflufenzopyr, Drechslera monoceras, endothal, ethofumesate, etobenzanid, fenoxasulfone, fentrazamide, flumiclorac-pentyl, flumioxazin, flupoxam, flurochloridone, flurtamone, indanofan, isoxaben, isoxaflutole, lenacil, propanil, propyzamide, quinclorac, quinmerac, mesotrione, methyl arsenic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, (3-[2-chloro-4-fluoro-5-(3-methyl-2,6-dioxo-4-trifluoromethyl-5 3,6-dihydro-2Hpyrimidin-1-yl)-phenoxy]-pyridin-2-yloxy)acetic acid ethyl ester, 6-amino-5-chloro-2-cyclopropyl-pyrimidine-4-carboxylic acid methyl ester, 6-chloro-3-(2-cyclopropyl-6-methylphenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-dimethylamino-2-fluoro-phenyl)-pyridine-2-carboxylic acid methyl ester.

In an embodiment, of the present invention, the pesticides are systemic pesticide.

In an embodiment of the present invention, the pesticides are selected from Fipronil, chlothianidin, Thiamethoxam, Acetamiprid, Dinotefuran, Imidacloprid, Thiacloprid, Flupyradifurone, Sulfoxaflor, Methiocarb, Tefluthrin, Bifenthrin, Cypermethrin Alphacypermethrin, Spinosad, Cyazypyr, Rynaxapyr, Thiodicarb, Triflumezopyrim (Mesoionic), Acephate, Chlorpyriphos, broflanilide, dimpropyridaz. In an embodiment of the present invention, the enzymes are selected from protease, peptidase, cellulase, hemicellulase, pectinase, chitinase, chitosanase, lipase, cutinase esterase, xylanase, and any combination thereof.

In yet another embodiment of the present invention, the enzyme is cutinase.

In an embodiment of the present invention, the biological source and the database/source of information is indicated in the below table A.

TABLE A

| AA | | | |
|---|---|---|---|
| Seq ID | Expression | Biological | Database/ |
| No. | Enzyme | Source | source |
| 1 | Cutinase 1 | Pseudomonas | USPTO |
| | | mendocina | US5545547A |
| 2 | Cutinase 2 | Pseudomonas | PDB 2FX5_A |
| | | mendocina | |
| 4 | Cutinase 4 | Pseudomonas | NCBI |
| | | mendocina | WP_096825365.1 |
| | | (94%) | |
| 9 | Cutinase 9 | Pseudomonas | NCBI |
| | | sp. SST3 | WP_168422850 |
| | | (90%) | |

In a further embodiment of the present invention the cutinase is Cutinase 1 which has the following sequence

SEQ NO. 1
APLPDTPGAPFPAVANFDRSGPYTTSSQSEGPSCRIYRPRDLGQGGVRHP
VILWGNGTGAGPSTYAGLLSHWASHGFVVAAAETSNAGTGREMLACLDYL
VRENDTPYGTYSGKLNTGRVGTSGHSQGGGGSIMAGQDTRVRTTAPIQPY
TLGLGHDSASQRRQQGPMFLMSGGGDTIAFPYLNAQPVYRRANVPVFWGE
RRYVSHFEPVGSGGAYRGPSTAWFRFQLMDDQDARATFYGAQCSLCTSLL
WSVERRGL

In a further embodiment of the present invention the cutinase is Cutinase 2 which has the following sequence

SEQ NO. 2
APLPDTPGAPFPAVANFDRSGPYTVSSQSEGPSCRIYRPRDLGQGGVRHP
VILWGNGTGAGPSTYAGLLSHWASHGFVVAAAETSNAGTGREMLACLDYL
VRENDTPYGTYSGKLNTGRVGTSGHSQGGGGSIMAGQDTRVRTTAPIQPY
TLGLGHDSASQRRQQGPMFLMSGGGDTIAFPYLNAQPVYRRANVPVFWGE
RRYVSHFEPVGSGGAYRGPSTAWFRFQLMDDQDARATFYGAQCSLCTSLL
WSVERRGL

In yet another embodiment of the present invention the cutinase is Cutinase 4 which has the following sequence

SEQ NO. 4
APLPDTPGAPLPAVANFDRSGPYATSNQSEGPSCRIYRPSNLGQGGVRHP
VILWGNGTGTGPSTYAGLLSHWASHGFVVAAAETSNAGTGREMLACLDYL
VRENDNPYGTYAGKLNTGRVGTSGHSQGGGGSIMAGQDTRVRTTAPIQPY
TIGLGHDSASQRRQQGPMFLMSGGGDTIAIPYLNAQPVYLRANVPVFWGE
RRYVSHFEPVGDGGAYRGPSTAWFRFQLMDDQSARGTFYGTLCSLCSSLL
WSVERRGF

In yet another embodiment of the present invention the cutinase is Cutinase 9 which has the following sequence

SEQ NO. 9
APLPDTPGAP
FPSVSNFDRSGPYATTSRSEGPNCRVYRPATLGQNGVRHPIVLWGNGTGT
GPTAYSGLLSHWASHGFVVAAAETSNAGTGEQMLACLDYLVQESNRTYGT
YVGVLNTGRVGTSGHSQGGGGSIMAGQDERVSVTAPIQPYTIGLGHDSAS
QRNQQGPMFLMSGGGDTIAFPYLNAQPVYTRANVPVFWGERRYVSHFEPV
GDGGAYRGPSTAWFRYHLMEDETARSTFYGRFCGLCTSVLWTEERKDIE

In an embodiment of the present invention, the nucleic acid sequence having at least 70%, at least 75%, at least 80%, at least 85%, at least 90%, at least 95%, at least 98%, at least 99%, or 100% identity to any one of SEQ ID NOs. 1,2,4,9.

In an embodiment of the present invention, the nucleic acid sequence having at least 70%, at least 75%, at least 80%, at least 85%, identity to any one of SEQ ID NOs. 1,2,4,9.

In an embodiment of the present invention, the nucleic acid sequence having at least 85%, identity to any one of SEQ ID NOs. 1,2,4,9.

In an embodiment of the present invention, the nucleic acid sequence having at least 85%, identity to any one of SEQ ID NOs. 1.

In an embodiment of the present invention, the nucleic acid sequence having at least 85%, identity to any one of SEQ ID NOs. 2.

In an embodiment of the present invention, the nucleic acid sequence having at least 85%, identity to any one of SEQ ID NOs. 4.

In an embodiment of the present invention, the nucleic acid sequence having at least 85%, identity to any one of SEQ ID NOs. 9.

In an embodiment, the enzyme may be used as a solution or as an immobilized enzyme.

In a further embodiment, any of the enzymes described herein can be immobilized on a matrix, support, or particle.

For example, the matrix, support, or particle can comprise charcoal, biochar, nanocarbon, agarose, an alginate, cellulose, a cellulose derivative, perlite, silica, plastic, stainless steel, glass, polystyrene, a ceramic, dolomite, a clay, diatomaceous earth, talc, a polymer, a gum, a water-dispersible material, or a combination of any thereof.

Immobilizing the enzyme on the matrix or support can result in a slower release of the enzyme as compared to the release rate for the same non-immobilized enzyme under the same conditions.

Use

In an embodiment, the presently claimed invention is directed to the use of at least one active compound and at least one enzyme for protecting plants or plant propagation material from attack or infestation by insects, fungi, arachnids or nematodes or protection against weeds, wherein the at least one active compound and the at least one enzyme is applied directly and/or indirectly to the plant and/or to plant propagation material by drenching the soil, by drip application onto the soil, by soil injection, by dipping, in-furrow application or by treatment of plant propagation material.

In an embodiment of the present invention, the at least one active compound and the at least one enzyme is applied directly and/or indirectly to the plant and/or to plant propagation material by drenching the soil.

In a further embodiment of the present invention, the at least one active compound and the at least one enzyme is applied directly and/or indirectly to the plant and/or to plant propagation material by drip application on the soil.

In yet another embodiment of the present invention, the at least one active compound and the at least one enzyme is applied directly and/or indirectly to the plant and/or to plant propagation material by soil injection.

In another embodiment of the present invention, the at least one active compound and the at least one enzyme is applied directly and/or indirectly to the plant and/or to plant propagation material by dripping.

In a further embodiment of the present invention, the at least one active compound and the at least one enzyme is applied directly and/or indirectly to the plant and/or to plant propagation material by in-furrow application.

In yet another embodiment of the present invention, the at least one active compound and the at least one enzyme is applied directly and/or indirectly to the plant and/or to plant propagation material by treatment of the plant propagation material.

In another embodiment of the present invention, the at least one active compound and the at least one enzyme is applied directly and/or indirectly to the plant and/or to plant propagation material by soaking the plant propagation material.

In an embodiment of the present invention, the at least one active compound which is a herbicide and the at least one enzyme is applied directly and/or indirectly to the plant and/or to plant propagation material to kill plants or inhibit root/shoot growth which is helpful with pre-emergence or early season herbicide application.

The active compound and the enzyme can be applied as granular as T-Band or in-furrow treatments.

Granular or liquid T-Band applications are placed in front of the furrow closure wheels using plastic diffusers. In general, the band coverage pattern is approximately a couple of inches wide over an open furrow.

In furrow treatments are directed into the open furrow using plastic tubing.

Liquid formulations are applied as T-band over an open furrow.

For example, seeds are planted using cone seeders and drop nozzles are positioned over the seed furrow. The boom can be moved up or down to change band width. A flat fan nozzle can also be used: perpendicular to the row for bands and parallel to the row for in-furrow. The boom position is between the furrow opener and the press wheel which directs some (band) or a (in-furrow) of the spray into the furrow before furrow closure. When used in in-furrow applications, the active compound(s) can be applied simultaneously with the planting of the seeds, e.g. as granular, liquid or another formulation type. Alternatively, the nozzles can also be positioned behind the press wheel for an entirely surface spray of the liquid formulation comprising the active compound(s).

The active compounds of the present invention are also suitable for the treatment of seeds in order to protect the seed from insect pest, in particular from soil-living insect pests and the resulting plant's roots and shoots against soil pests and foliar insects.

The active compounds of the present invention are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedling's roots and shoots from insects, in particular from soil and foliar insects, said method comprising contacting the seeds before sowing and/or after pre-germination with an active compounds of the present invention. Particularly preferred is a method, wherein the plant's roots and shoots are protected, more preferably a method, wherein the plants shoots are protected from piercing and sucking insects, most preferably a method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Crops

The present invention relates to the methods by use on natural substrates (soil) or artificial (growth) substrates (e.g. rock wool, glass wool, quartz sand, gravel, expanded clay, vermiculite), in the open or in closed systems (e.g. greenhouses or under film mulch) and in annual crops (such as vegetables, spices, ornamentals) or perennial crops (such as citrus plants, fruits, tropical crops, spices, nuts, grapevines, conifers and ornamentals).

It has now been found that the problems associated with combating soil-living pests by pesticide treatment of the soil can be overcome by such application methods using compounds described in the present invention.

The animal pest, i.e. the insects, arachnids and nematodes, the plant, the water or the soil in which the plant is growing can be contacted with the active compounds described in the present invention containing them by any application method known in the art. As such, “contacting” includes both direct contact (applying the compounds/compositions directly on the animal pest or plant) and indirect contact (applying the compounds/compositions to the locus of the animal pest or plant). When the plant is contacted, typically the tuber, bulbs or roots of the plant are contacted. The active compounds of the present invention may further be applied to other parts of the plant, such as leaves in case of foliar application, or to plant propagation material such as seeds in the case of seed treatment.

The active compounds described in the present invention or the pesticidal compositions comprising them may be used to protect growing plants and crops from attack or infestation by animal pests, especially insects, acaridae or arachnids by contacting the plant/crop with a pesticidally effective amount of active compounds of the present invention. The term “crop” refers both to growing and harvested crops.

Thus, as with regards to the use and for the purpose of the present invention, vegetables are to be understood as meaning for example fruiting vegetables and inflorescences as vegetables, i.e. be peppers, chillies, tomatoes, aubergines, cucumbers, pumpkins, courgettes, broad beans, climbing and dwarf beans, peas, artichokes and maize. Further also leafy vegetables like head-forming lettuce, chicory, endives, various types of cress, of rocket, lamb's lettuce, iceberg lettuce, leeks, spinach and chard. Furthermore, tuber vegetables, root vegetables and stem vegetables, like celeriac/celery, beetroot, carrots, radish, horseradish, scorzonera, asparagus, beet for human consumption, palm hearts and bamboo shoots. Further also bulb vegetables like onions, leeks, fennel and garlic. Brassica vegetables such as cauliflower, broccoli, kohlrabi, red cabbage, white cabbage, curly kale, Savoy cabbage, Brussels sprouts and Chinese cabbage are also vegetable in the sense of the present application.

Regarding the use and for the purpose of the present invention, perennial crops are to be understood as meaning citrus, for example, oranges, grapefruits, tangerines, lemons, limes, Seville oranges, cumquats and satsumas. Also, pome fruit such as, for example, apples, pears and quinces, and stone fruit such as, for example, peaches, nectarines, cherries, plums, quetsch, apricots. Further grapevines, hops, olives, tea and tropical crops such as, for example, mangoes, papayas, figs, pineapples, dates, bananas, durians, kaki fruit, coconuts, cacao, coffee, avocados lychees, maracujas, and. guavas. Furthermore, soft fruit such as, for example, currants, gooseberries, raspberries, blackberries, blueberries, strawberries, cranberries, kiwi fruit and American cranberries. Almonds and nuts such as, for example, hazelnuts, walnuts, pistachios, cashew nuts, para nuts, pecan nuts, butternuts, chestnuts, hickory nuts, macadamia nuts and peanuts are also fruits in the sense of the present invention.

As with regard to the use and for the purpose of the present invention, ornamentals are understood as meaning annual and perennial plants, for example cut flowers such as, for example, roses, carnations, gerbera, lilies, marguerites, chrysanthemums, tulips, narcissus, anemones, poppies, amaryllis, dahlias, azaleas, hibiscus, but also for example border plants, pot plants and perennials such as, for example, roses, Tagetes, violas, geraniums, fuchsias, hibiscus, chrysanthemum, busy lizzie, cyclamen, African violet, sunflowers, begonias.

Furthermore, for example also bushes and conifers such as, for example, ficus, rhododendron, firs, spruces, pines, yews, juniper, umbrea pines, oleander.

As regards the use, spices are understood as meaning annual and perennial plants such as, for example, aniseed, chilli pepper, paprika, pepper, vanilla, marjoram, thyme, cloves, juniper berries, cinnamon, tarragon, coriander, saffron, ginger. Furthermore the compounds of the present invention and the compositions comprising them are particularly important in the control of a multitude of insects on various cultivated plants, such as cereal and oil crops, for example seed of durum and other wheat, barley, oats, rye, maize (fodder maize and sugar maize/sweet and field corn), soybeans, oil crops, crucifers, cotton, bananas, rice, oilseed rape, turnip rape, sugarbeet, fodder beet, eggplants, potatoes, grass, lawn, turf, fodder grass, sugar cane or tobacco.

The compounds of the invention can also be applied preventively to places at which occurrence of the pests is expected. “Locus” means a habitat, breeding ground, plant, seed, soil, area, material or environment in which a pest or parasite is growing or may grow.

The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “cultivated plants” is to be understood as including plants which have been modified by breeding, mutagenesis or genetic engineering. Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transitional modification of protein(s) (oligo- or polypeptides) poly for example by glycosylation or polymer additions such as prenyl-ated, acetylated or farnesylated moieties or PEG moieties (e.g. as disclosed in Biotechnol Prog. 2001 July-August; 17(4):720-8, Protein Eng Des Sel. 2004 January; 17(1):57-66, Nat Protoc. 2007; 2(5):1225-35, Curr Opin Chem Biol. 2006 October; 10(5):487-91. Epub 2006 Aug. 28, Bio-materials. 2001 March; 22(5):405-17, Bioconjug Chem. 2005 January-February; 16(1):1 13-21.

The term “cultivated plants” is to be understood also including plants that have been rendered tolerant to applications of specific classes of herbicides, such as hydroxy-phenylpyruvate dioxygenase (HPPD) inhibitors; acetolactate synthase (ALS) inhibitors, such as sulfonyl ureas (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073); enolpyruvylshikimate-3-phosphate synthase (EPSPS) inhibitors, such as glyphosate (see e.g. WO 92/00377); glutamine synthetase (GS) inhibitors, such as glufosinate (see e.g. EP-A-0242236, EP-A-242246) or oxynil herbicides (see e.g. U.S. Pat. No. 5,559,024) as a result of conventional methods of breeding or genetic engineering. Several cultivated plants have been rendered tolerant to herbicides by conventional methods of breeding (mutagenesis), for example Clearfield® summer rape (Canola) being tolerant to imidazolinones, e.g. imazamox. Genetic engineering methods have been used to render cultivated plants, such as soybean, cotton, corn, beets and rape, tolerant to herbicides, such as glyphosate and glufosinate, some of which are commercially available under the trade names RoundupReady® (glyphosate) and LibertyLink® (glufosinate).

The term “cultivated plants” is to be understood also including plants that are by the use of recombinant DNA techniques capable to synthesize one or more insecticidal proteins, especially those known from the bacterial genus Bacillus, particularly from Bacillus thuringiensis, such as a-endotoxins, e.g. CryIA(b), CryIA(c), CryIF, CryIF(a2), CryIIA(b), CryIIIA, CryIIIB(bI) or Cry9c; vegetative insecticidal proteins (VIP), e.g. VIP1, VIP2, VIP3 or VIP3A; insecticidal proteins of bacteria colonizing nematodes, for example Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pretoxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are dis-closed, for ex-ample, in EP-A 374 753, WO 93/007278, WO 95/34656, EP-A 427 529, EP-A 451 878, WO 03/018810 und WO 03/052073. The methods for producing such genetically modified plants are generally known to the person skilled in the art and are described, for example, in the publications mentioned above. These insecticidal proteins contained in the genetically modified plants impart to the plants producing these proteins protection from harmful pests from certain taxo-nomic groups of arthropods, particularly to beetles (Coleoptera), flies (Diptera), and butterflies and moths (Lepidoptera) and to plant parasitic nematodes (Nematoda).

The term “cultivated plants” is to be understood also including plants that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, for example EP-A 0 392 225), plant disease resistance genes (for example potato cultivars, which express resistance genes acting against Phytophthora infestans derived from the mexican wild potato So-lanum bulbocastanum) or T4-lyso-zym (e.g. potato cultivars capable of synthesizing these proteins with increased resistance against bacteria such as Erwinia amylvora). The methods for producing such genetically modified plants are generally known to the person skilled in the art and are described, for example, in the publications mentioned above.

The term “cultivated plants” is to be understood also including plants that are by the use of recombinant DNA techniques capable to synthesize one or more proteins to increase the productivity (e.g. bio mass production, grain yield, starch content, oil content or protein content), tolerance to drought, salinity or other growth-limiting environ-mental factors or tolerance to pests and fungal, bacterial or viral pathogens of those plants. The term “cultivated plants” is to be understood also including plants that contain by the use of recombinant DNA techniques a modified amount of substances of content or new substances of content, specifically to improve human or animal nutrition, for ex-ample oil crops that produce health-promoting long-chain omega-3 fatty acids or unsaturated omega-9 fatty acids (e.g. Nexera® rape).

The term “cultivated plants” is to be understood also including plants that contain by the use of recombinant DNA techniques a modified amount of substances of content or new substances of content, specifically to improve raw material production, for example potatoes that produce increased amounts of amylopectin (e.g. Amflora® potato) In an embodiment of the present invention suitable seed are seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In an embodiment of the present invention the method or use of protecting the plants or plant propagation material from attack or infestation by the following pests.

The invention in particular relates to soil application methods for combating soil-living arthropod pests, and nematode pests, which comprises applying to the soil a pesticidally effective amount of a compound of the present invention. The term “soil-living” means that the habitat, breeding ground, area or environment in which a pest or parasite is growing or may grow is the soil.

The use of the compounds according to the present invention extends to a wide range of different animal pests, especially soil living pests. These include but are not limited to, the following families:

Insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellulaundalis, Hibemia defoliaria, Hyphantria cunea, Hyponomeuta malinellus, Keiferia lycopersicella, Lambdina fiscellana, Laphygma exigua, Leucoptera coffeella, Leucoptera scitella, Lithocolletis blancardella, Lobesia botrana, Loxostege sticticalis, Lymantria dispar, Lymantria monacha, Ly-onetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xy-lostella, Pseudoplusia includens, Rhyacionia frustrana, Scrobipalpula absoluta, Sitotroga cere-alella, Sparganothis pilleriana, Spodoptera frugiperda, Spodoptera littoralis, Spodoptera litura, Thaumatopoea pityocampa, Tortrix viridana, Trichoplusia ni, Tuta absoluta, and Zeiraphera canadensis, beetles (Coleoptera), for example Agrilus sinuatus, Agriotes lineatus, Agriotes obscurus, Am-phimallus solstitialis, Anisandrus dispar, Anoplophora glabripensis, Anthonomus grandis, An-thonomus pomorum, Aphthona euphoridae, Athous haemorrhoidalis, Atomaria linearis, Blas-tophagus piniperda, Blitophaga undata, Byctiscus betulae, Cassida nebulosa, Cerotoma trifur-cata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica punctata Diabrotica speciosa, Diabrotica virgifera, Epilachna varivestis, Epitrix hirtipennis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Otiorrhynchus sulcatus, Otiorrhynchus ovatus, Phae-don cochleariae, Phyllobius pyri, Phyllotreta chrysocephala, Phyllophaga sp., Phyllopertha hor-ticola, Phyllotreta nemorum, Phyllotreta striolata, Popillia japonica, Sitona lineatus and, flies, mosquitoes (Diptera), e.g. Ceratitis capitata, Contarinia sorghicola Dacus cucurbitae, Dacus oleae, Dasineura brassicae, Delia antique, Delia coarctata, Delia platura, Delia radicum, Liriomyza sativae, Liriomyza trifolii, Oscinella frit, Pegomya hysocyami, Phorbia antiqua, Phor-bia brassicae, Phorbia coarctata, Psila rosae, Psorophora discolor, Rhagoletis cerasi, Rhago-letis pomonella, Tipula oleracea, and Tipula paludosa thrips (Thysanoptera), e.g. Dichromothrips corbetti, Dichromothrips ssp., Frankliniella fusca, Frankliniella occidentalis, Frankliniella tritici, Scirtothrips citri, Thrips oryzae, Thrips palmi and Thrips tabaci, termites (Isoptera), e.g. Calotermes flavicollis, Leucotermes flavipes, Heterotermes aureus, Reticulitermes flavipes, Reticulitermes virginicus, Reticulitermes lucifugus, Reticulitermes san-tonensis, Reticulitermes grassei, Termes natalensis, and Coptotermes formosanus, bugs, aphids, leafhoppers, whiteflies, scale insects, cicadas (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphispomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachy-caudus helichrysi, Brachycaudus persicae, Brachycaudus prunicola, Brevicoryne brassicae, Capitophorus horni, Cerosipha gossypii, Chaetosiphon fragaefoli, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Euschistos heros, Euschistos servus, Halyomor-pha halys, Hyalopterus pruni, Hyperomyzus lactucae, Macrosiphum avenae, Macrosiphum eu-phorbiae, Macrosiphon rosae, megacopta criberia, Megoura viciae, Melanaphis pyrarius, Metopolophium dirhodum, Myzus persicae, Myzus ascalonicus, Myzus cerasi, Myzus varians, Nasonovia ribis-nigri, Nezara viridula, Nilaparvata lugens, Pemphigus bursarius, Perkinsiella saccharicida, Phorodon humuli, Psylla mail, Psylla piri, Rhopalomyzus ascalonicus, Rhopalosiphum maidis, Rhopalosiphum padi, Rhopalosiphum insertum, Sappaphis mala, Sap-paphis mali, Schizaphis graminum, Schizoneura lanuginosa, Sitobion avenae, Trialeurodes va-porariorum, Toxoptera aurantiiand, Viteus vitifolii, Cimex lectularius, Cimex hemipterus, Reduvi-us senilis, Triatoma spp., and Arilus critatus. ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Lasius niger, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Polistes rubigi-nosa, Camponotus floridanus, and Linepithema humile, crickets, grasshoppers, locusts (Orthoptera), e.g. Acheta domestica, Gryllotalpa gryllotalpa, Lo-custa migratoria, Melanoplus bivittatus, Melanoplus femurrubrum, Melanoplus mexicanus, Mel-anoplus sanguinipes, Melanoplus spretus, Nomadacris septemfasciata, Schistocerca america-na, Schistocerca gregaria, Dociostaurus maroccanus, Tachycines asynamorus, Oedaleus sen-egalensis, Zonozerus variegatus, Hieroglyphus daganensis, Kraussaria angulifera, Calliptamus italicus, Chortoicetes terminifera, and Locustana pardalina,

Arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus mou-bata, Ornithodorus hermsi, Ornithodorus turicata, Ornithonyssus bacoti, Otobius megnini, Der-manyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus palli-dus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa, Other animal pests to be controlled and combated by the methods of the present invention are: From the family of the Pemphigidae: Eriosoma spp., Pemphigus spp., Anuraphis spp., Brachy-caudus spp., in crops such as, for example, pome fruit, conifers, vegetables and ornamentals.

From the psyllid family (Psyllidae: Psylla spp., Paratrioza spp., Trioza spp., in crops such as, for example, citrus, vegetables, potatoes, pome fruit.

From the scale insect family (Coccidae: Ceroplastes spp., Drosicha spp. Pulvinaria spp., Protopuhninaria spp., Saissetia spp., Coccus spp., in perennial crops such as, for example, citrus, grapevines, tea, pome and stone fruit, tropical crops, ornamentals, conifers, but also vegetables.

From the family of the Diaspididae: Quadraspidiotus spp., Aonidiella spp., Lepidosaphes spp., Aspidiotus spp., Aspis spp., Diaspis spp., Parlatoria spp., Pseudaulacaspis spp., Unaspis spp., Pinnaspis spp., Selenaspidus spp., in crops such as, for example, citrus, tea, ornamentals, conifers, pome and stone fruit, grapevines, tropical crops.

From the family of the Pseudococcidae: Pericerga, Pseudococcus spp., Planococcus spp., Phenacoccus spp., Dysmicoccus spp., in crops such as, for example, citrus, pome and stone fruit, tea, grapevines, vegetables, ornamentals, conifers, spices and tropical crops.

Furthermore from the family of the Aleyrodidae: Bemisia argentifolli, Bemisia tabaci, Trialeurodes vapora riorum, Aleurothrixus floccosus, Aleurodes spp., Dialeurodes spp., Parabemisia myricae in crops such as, for example, vegetables, melons, potatoes, tobacco, soft fruit, citrus, ornamentals, conifers, cotton, potatoes and tropical crops.

Furthermore, from the family of the Aphidae:

Myzus spp. in tobacco, stone fruit, pome fruit, soft fruit, Brassica vegetables, fruiting vegetables, leafy vegetables, tuber and root vegetables, melons, potatoes, spices, ornamentals and conifers.

Aphis spp. in cotton, tobacco, citrus, melons, beet, soft fruit, oilseed rape, fruiting vegetables, leafy vegetables, Brassica vegetables, tuber and root vegetables, ornamentals, potatoes, pumpkins, spices. Rhodobium porosum in strawberries,

Nasonovia ribisnigri in leafy vegetables,

Macrosiphum spp. in ornamentals, cereals, potatoes, leafy vegetables, Brassica vegetables and fruiting vegetables, strawberries, Phorodon humuli in hops, Toxoptera spp. in citrus, stone fruit, almonds, nuts, cereals, spices,

Aulacorthum spp. in citrus, potatoes, fruiting vegetables and leafy vegetables.

Furthermore the following from the family of the Tetranychidae: Tetranychus spp., Brevipalpus spp., Panonychus spp., Oligonycbus spp., Eotetranychus spp., Bryobia spp. in crops such as, for example, vegetables, ornamentals, spices, conifers, citrus, stone and pome fruit, grapevines, cotton, soft fruit, melons, potatoes. The following from the family of the Tarsonemidae: Hermitarsonemus batus, Stenotarsonemus spp., Polyphagotarsonemus spp., Stenotarsonemus spinki in crops such as, for example, vegetables, ornamentals, spices, conifers, tea, citrus, melons.

Furthermore the following from the thrips family (Thripidae): Anaphothrips spp., Baliothrips spp., Caliothrips spp., Frarikliruella spp., Heliothrips spp., Hercrnothrips spp., Rhipiphorothrips spp., Scirtothrips spp., Selenothrips spp. and Thrips spp., in crops such as, for example, fruit, cotton, grapevines, soft fruit, vegetables, melons, ornamentals, spices, conifers, tropical crops, tea. Also the following from the whitefly family (Agromyzidae): Liriomyza spp., Pegomya spp. in crops such as, for example, vegetables, melons, potatoes and ornamentals.

Also the following from the foliar nematode family (Aphelenchoididae), for example Aphelenchoides ritzemabosi, A. fragariae, A. besseyi, A. blastophthorus in crops such as soft fruits and ornamentals.

The methods of the present invention are applied to control and combat arachnids, especially the following ones from the family of the Tetranychidae: Tetranychus spp., Brevipalpus spp., Panonychus spp., Oligonycbus spp., Eotetranychus spp. and Bryobia spp.

Fungicidal active ingredients in the methods according to the present invention, are also especially suitable for efficiently combating phytopathogenic fungi.

They have excellent activity against a broad spectrum of phytopathogenic fungi Ascomycetes, Basidiomycetes, Deuteromycetes and Peronosporomycetes (syn. Oomycetes). Some of them are systemically effective and can be employed in crop protection as foliar fungicides, as fungicides for seed dressing and as soil fungicides. They can also be used for treating seed.

They are particularly important in the control of a multitude of fungi on various cultivated plants, such as wheat, rye, barley, oats, rice, corn, lawns, bananas, cotton, soybean, coffee, sugar cane, grapevines, fruits and ornamental plants, and vegetables such as cucumbers, beans, tomatoes, potatoes and cucurbits, and on the seeds of these plants.

They are especially suitable for controlling the following plant diseases:

Albugo spp. (white rust) on ornamentals, vegetables (e.g. A. Candida) and sunflowers (e.g. A. tragopogonis); Alternaria spp. (Alternaria leaf spot) on vegetables, rape (A. brassicola or brasss′-cae), sugar beets (A. tenuis), fruits, rice, soybeans, potatoes (e.g. A. solani or A. alternata), tomatoes (e.g. A. solani or A. alternata) and wheat; Aphanomyces spp. on sugar beets and vegetables; Ascochyta spp. on cereals and vegetables, e.g. A. tritici (anthracnose) on wheat and A. hordei on barley; Bipolaris and Drechslera spp. (teleomorph: Cochliobolus spp.), e.g. Southern leaf blight (D. maydis) or Northern leaf blight (B. zeicola) on corn, e.g. spot blotch (B. sorokiniana) on cereals and e.g. B. oryzae on rice and turfs; Blumeria (formerly Erysiphe) graminis (powdery mildew) on cereals (e.g. on wheat or barley); Botrytis cinerea (teleomorph: Botryotinia fuckeliana: grey mold) on fruits and berries (e.g. strawberries), vegetables (e.g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e.g. C. ulmi (Dutch elm disease) on elms; Cercospora spp. (Cer-cospora leaf spots) on corn (e.g. Gray leaf spot: C. zeae-maydis), rice, sugar beets (e.g. C. beticola), sugar cane, vegetables, coffee, soybeans (e.g. C. sojina or C. kikuchii) and rice; Cladosporium spp. on tomatoes (e.g. C. fulvum: leaf mold) and cereals, e.g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e.g. C. sativus, anamorph: B. sorokiniana) and rice (e.g. C. miyabeanus, anamorph: H. oryzae); Colletotrichum (teleomorph: Glomerella) spp. (anthracnose) on cotton (e.g. C. gossypii), corn (e.g. C. gramini-co/a; Anthracnose stalk rot), soft fruits, potatoes (e.g. C. coccodes: black dot), beans (e.g. C. lindemuthianum) and soybeans (e.g. C. truncatum or C. gloeosporioides); Corticium spp., e.g. C. sasakii (sheath blight) on rice; Corynespora cassiicola (leaf spots) on soybeans and ornamentals; Cycloconium spp., e.g. C. oleaginum on olive trees; Cylindrocarpon spp. (e.g. fruit tree canker or young vine decline, teleomorph: Nectria or Neonectria spp.) on fruit trees, vines (e.g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e.g. D. phaseolorum (damping off) on soybeans; Drechslera (syn. Helminthosporium, teleomorph: Pyrenophora) spp. on corn, cereals, such as barley (e.g. D. teres, net blotch) and wheat (e.g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (£. pyri), soft fruits (£. veneta: anthracnose) and vines (£. ampelina: anthracnose); Entyloma oryzae (leaf smut) on rice; Epicoccum spp. (black mold) on wheat; Erysiphe spp. (powdery mildew) on sugar beets (£. betae), vegetables (e.g. E. pisi), such as cucurbits (e.g. E. cichoracearum), cabbages, rape (e.g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e.g. E. turci-cum); Fusarium (teleomorph: Gibberella) spp. (wilt, root or stem rot) on various plants, such as F. graminearum or F. culmorum (root rot, scab or head blight) on cereals (e.g. wheat or barley), F. oxysporum on tomatoes, F. solani (f. sp. glycines now syn. F. virguliforme) and F. tucumani-ae and F. brasiliense each causing sudden death syndrome on soybeans, and F. verticillioides on corn; Gaeumannomyces graminis (take-all) on cereals (e.g. wheat or barley) and corn; Gibberella spp. on cereals (e.g. G. zeae) and rice (e.g. G. fujikuroi: Bakanae disease); Glomerella cingulata on vines, pome fruits and other plants and G. gossypii on cotton; Grainstaining com-plex on rice; Guignardia bidwellii (black rot) on vines; Gymnosporangium spp. on rosaceous plants and junipers, e.g. G. sabinae (rust) on pears; Helminthosporium spp. (syn. Drechslera, teleomorph: Cochliobolus) on corn, cereals and rice; Hemileia spp., e.g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e.g. wheat or barley); Microsphaera diffusa (powdery mildew) on soybeans; Monilinia spp., e.g. M. laxa, M. fructicola and M. fructigena (bloom and twig blight, brown rot) on stone fruits and other rosaceous plants; Mycosphaerella spp. on cereals, bananas, soft fruits and ground nuts, such as e.g. M. graminicola (anamorph: Septoria tritici, Septoria blotch) on wheat or M. fijiensis (black Sigatoka disease) on bananas; Peronospora spp. (downy mildew) on cabbage (e.g. P. brassicae), rape (e.g. P. parasitica), onions (e.g. P. destructor), tobacco (P. tabacina) and soybeans (e.g. P. manshurica); Phakopsora pachyrhizi and P. meibomiae (soybean rust) on soybeans; Phialophora spp. e.g. on vines (e.g. P. tracheiphila and P. tetraspora) and soybeans (e.g. P. gregata: stem rot); Phoma lingam (root and stem rot) on rape and cabbage and P. betae (root rot, leaf spot and damping-off) on sugar beets; Phomopsis spp. on sunflowers, vines (e.g. P. viticola: can and leaf spot) and soybeans (e.g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e.g. P. capsici), soybeans (e.g. P. megasperma, syn. P. sojae), potatoes and tomatoes (e.g. P. infestans: late blight) and broadleaved trees (e.g. P. ramorum: sudden oak death); Plasmodiophora brassicae (club root) on cabbage, rape, radish and other plants; Plasmopara spp., e.g. P. viticola (grapevine downy mildew) on vines and P. halstedii on sunflowers; Podosphaera spp. (powdery mildew) on rosaceous plants, hop, pome and soft fruits, e.g. P. leucotricha on apples; Polymyxa spp., e.g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e.g. wheat or barley; Pseudoperonospora (downy mildew) on various plants, e.g. P. cubensis on cucurbits or P. humili on hop; Pseudopezicula tracheiphila (red fire disease or .rotbrenner’, anamorph: Phialophora) on vines; Puccinia spp. (rusts) on various plants, e.g. P. triticina (brown or leaf rust), P. striiformis (stripe or yellow rust), P. hordei (dwarf rust), P. graminis (stem or black rust) or P. recondita (brown or leaf rust) on cereals, such as e.g. wheat, barley or rye, P. kuehnii (orange rust) on sugar cane and P. asparagi on asparagus; Pyrenophora (anamorph: Drechslera) tritici-repentis (tan spot) on wheat or P. feres (net blotch) on barley; Pyricularia spp., e.g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e.g. P. ultimum or P. aphanidermatum); Ramularia spp., e.g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e.g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sa-rocladium oryzae and S. attenuatum (sheath rot) on rice; Sclerotinia spp. (stem rot or whitemold) on vegetables and field crops, such as rape, sunflowers (e.g. S. sclerotiorum) and soybeans (e.g. S. rolfsii or S. sclerotiorum); Septoria spp. on various plants, e.g. S. glycines (brown spot) on soybeans, S. tritici (Septoria blotch) on wheat and S. (syn. Stagonospora) no-dorum (Stagonospora blotch) on cereals; Uncinula (syn. Erysiphe) necator (powdery mildew, anamorph: Oidium tuckeri) on vines; Setospaeria spp. (leaf blight) on corn (e.g. S. turcicum, syn. Helminthosporium turcicum) and turf; Sphacelotheca spp. (smut) on corn, (e.g. S. reiliana: head smut), sorghum und sugar cane; Sphaerotheca fuliginea (powdery mildew) on cucurbits; Spongospora subterranea (powdery scab) on potatoes and thereby transmitted viral diseases; Stagonospora spp. on cereals, e.g. S. nodorum (Stagonospora blotch, teleomorph: Lepto-sphaeria [syn. Phaeosphaeria] nodorum) on wheat; Synchytrium endobioticum on potatoes (potato wart disease); Taphrina spp., e.g. T. deformans (leaf curl disease) on peaches and T. pruni (plum pocket) on plums; Thielaviopsis spp. (black root rot) on tobacco, pome fruits, vegetables, soybeans and cotton, e.g. T. basicola (syn. Chalara elegans); Tilletia spp. (common bunt or stinking smut) on cereals, such as e.g. T. tritici (syn. T. caries, wheat bunt) and T. controversa (dwarf bunt) on wheat; Typhula incarnata (grey snow mold) on barley or wheat; Urocystis spp., e.g. U. occulta (stem smut) on rye; Uromyces spp. (rust) on vegetables, such as beans (e.g. U. appendiculatus, syn. U. phaseoli) and sugar beets (e.g. U. betae); Ustilago spp. (loose smut) on cereals (e.g. U. nuda and U. avaenae), corn (e.g. U. maydis: corn smut) and sugar cane; Venturia spp. (scab) on apples (e.g. V. inaequalis) and pears; and Verticillium spp. (wilt) on various plants, such as fruits and ornamentals, vines, soft fruits, vegetables and field crops, e.g. V. dahliae on strawberries, rape, potatoes and tomatoes.

The compositions of the presently claimed invention are suitable for controlling a large number of harmful plants, including monocotyledonous weeds and dicotyledonous weeds. They are in particular useful for controlling annual weeds such as gramineous weeds (grasses) including Echinochloa species such as barnyardgrass (Echinochloa crusgalli var. crusgalli), Digitaria species such as crabgrass (Digitaria sanguinalis), Setaria species such as green foxtail (Setaria vindis) and giant foxtail (Setaria faberii), Sorghum species such as johnsongrass (Sorghum halepense Pers.), Avena species such as wild oats (Avena fatua), Cenchrus species such as Cenchrus echinatus, Bromus species, Lolium species, Phalaris species, Eriochloa species, Panicum species, Brachiaria species, annual bluegrass (Poa annua), blackgrass (Alopecurus myosuroides), Aegilops cylindrica, Agropyron repens, Apera spicaventi, Eleusine indica, Cynodon dactylon and the like. The compositions of the presently claimed invention are also suitable for controlling a large number of dicotyledonous weeds, in particular broad leaf weeds including particular broadleaf weeds including Polygonum species such as wild buckwheat (Polygonum convolvolus), Amaranthus species such as pigweed (Amaranthus retroflexus), Chenopodium species such as common lambsquarters (Chenopodium album L.), Sida species such as prickly sida (Sida spinosa L.), Ambrosia species such as common ragweed (Ambrosia artemisllfolia), Acanthospermum species, Anthemis species, Atriplex species, Cirsium species, Convolvulus species, Conyza species, such as horseweed (Conyza canadensis), Cassia species, Commelina species, Datura species, Euphorbia species, Geranium species, Galinsoga species, morningglory (Ipomoea species), Lamium species, Malva species, Matricaria species, Sysimbrium species, Solanum species, Xanthium species, Veronica species, Viola species, common chickweed (Stellaria media), velvetleaf (Abutilon theophrasti), Hemp sesbania (Sesbania exaltata Cory), Anoda cristata, Bidens pllosa, Brassica kaber, Capsella bursa-pastoris, Centaurea cyanus, Galeopsis tetrahit, Galium aparine, Helianthus annuus, Desmodium tortuosum, Kochia scoparia, Mercurialis annua, Myosotis arvensis, Papaver rhoeas, Raphanus raphanistrum, Salsola kali, Sinapis arvensis, Sonchus arvensis, Thlaspi arvense, Tagetes minuta, Richardia braslliensis, and the like

Composition

In a further embodiment, the presently claimed invention is directed to a composition for protecting plants or plant propagation material from attack or infestation by insects, fungi, arachnids or nematodes or protection against weeds, comprising:

at least one active compound and at least one enzyme selected from the group consisting of protease, peptidase, cellulase, hemiceulase, pectinase, chitinase, chitosanase, lipase, cutinase esterase, xylanase.

An agrochemical composition comprises a pesticidally effective amount of active compound present invention. The term “effective amount” denotes an amount of the composition or of the compounds I, which is sufficient for controlling harmful pests on cultivated plants or in the protection of materials and which does not result in a substantial damage to the treated plants. Such an amount can vary in a broad range and is dependent on various factors, such as the animal pests species to be controlled, the treated cultivated plant or material, the climatic conditions and the specific active compound of the present invention.

The active compounds of the present invention, their N-oxides and salts can be converted into customary types of agrochemical compositions, e.g. solutions, emulsions, suspensions, dusts, powders, pastes, granules, pressings, capsules, and mixtures thereof. Examples for composition types are sus-pensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6th Ed. May 2008, CropLife International. The compositions are prepared in a known manner, such as described by Mollet and Grube mann, Formulation technology, Wiley VCH, Weinheim, 2001; or Knowles, New developments in crop protection product formulation, Agrow Reports DS243, T&F Informa, London, 2005. Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders. Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, non-ionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyhnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable non-ionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkyl-polyglycosides. Examples of polymeric surfactants are homo- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or poly-ethyleneamines.

Suitable adjuvants are compounds, which have a neglectable or even no pesticidal activity themselves, and which improve the biological performance of the active compound of the present invention on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), inorganic clays (organically modified or unmodified), polycarboxylates, and silicates.

Suitable bactericides are bronopol and isothiazolinone derivatives such as alkylisothiazolinones and benzisothiazolinones. Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidone, polyvinylacetates, polyvinyl alcohols, polyacrylates, biological or synthetic waxes, and cellulose ethers.

The agrochemical compositions generally comprise between 0.01 and 95%, preferably between 0.1 and 90%, and in particular between 0.5 and 75%, by weight of active substance. The active substances are employed in a purity of from 90% to 100%, preferably from 95% to 100% (according to NMR spectrum).

Solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying the compounds of the present invention and compositions thereof, respectively, on to plant propagation material, especially seeds include dressing, coating, pelleting, dusting, soaking and in furrow application methods of the propagation material. Preferably, compound of the present invention or the compositions thereof, respectively, are applied on to the plant propagation material by a method such that germination is not induced, e.g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a pre-dosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 litres, preferably 50 to 400 litres, of the ready-to-use spray liquor are applied per hectare of agricultural useful area.

Compositions which are especially useful for seed treatment are e.g.:
A Soluble concentrates (SL, LS)
D Emulsions (EW, EO, ES)
E Suspensions (SC, OD, FS)
F Water-dispersible granules and water-soluble granules (WG, SG)
G Water-dispersible powders and water-soluble powders (WP, SP, WS)
H Gel-Formulations (GF)
I Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter.

In a preferred embodiment a FS formulation is used for seed treatment. Typically, a FS formulation may comprise 1-800 g/l of active ingredient, 1-200 g/l Surfactant, 0 to 200 g/l anti-freezing agent, 0 to 400 g/l of binder, 0 to 200 g/l of a pigment and up to 1 litre of a solvent, preferably water.

Especially preferred FS formulations of active compound of the present invention for seed treatment usually comprise from 0.1 to 80% by weight (1 to 800 g/1) of the active ingredient, from 0.1 to 20% by weight (1 to 200 g/1) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed Treatment formulations may additionally also comprise binders and optionally colorants. Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, polysaccharides like celluloses, tylose and starch, polyolefin homo- and copolymers like olefin/maleic anhydride copolymers, polyurethanes, polyesters, polystyrene homo and copolymers.

Optionally, also colorants can be included in the formulation. Suitable colorants or dyes for seed treatment formulations are Rhodamin B, C.I. Pigment Red 1 12, C.I. Solvent Red 1, pigment blue 15:4, pigment blue 15:3, pigment blue 15:2, pigment blue 15:1, pigment blue 80, pigment yellow 1, pigment yellow 13, pigment red 1 12, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®).

In the treatment of seed, the application rates of the compound of the present invention are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed.

The invention therefore also relates to seed comprising an active compound of the present invention, or an agriculturally useful salt of I, as defined herein. The amount of the active compound or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

In yet another embodiment, the presently claimed invention is directed to seeds comprising at least one active compound and at least one enzyme selected from the group consisting of protease, peptidase, cellulase, hemicellulase, pectinase, chitinase, chitosanase, lipase, cutinase esterase and xylanase in an amount of from 1 ppb to 1 ppt per weight of seeds or plant propagation medium.

In an embodiment the enzyme is cutinase, wherein the cutinase of SEQ no. 1 is applied in an amount of from 1 ppb to 1 ppt per weight of the seeds or the plant propagation material.

In an embodiment the enzyme is cutinase wherein the cutinase of SEQ no. 2 is applied in an amount of from 1 ppb to 1 ppt per weight of the seeds or the plant propagation material.

In an embodiment the enzyme is cutinase, wherein the cutinase of SEQ no. 4 is applied in an amount of from 1 ppb to 1 ppt per weight of the seeds or the plant propagation material.

In an embodiment the enzyme is cutinase, wherein the cutinase of SEQ no. 9 is applied in an amount of from 1 ppb to 1 ppt per weight of the seeds or the plant propagation material.

In an embodiment the enzyme is cutinase, wherein the cutinase of SEQ no. 1 is applied in an amount of from 1 ppm to 1 ppb per weight of the seeds or the plant propagation material.

In an embodiment the enzyme is cutinase, wherein the cutinase of SEQ no. 2 is applied in an amount of from 1 ppm to 1 ppb per weight of the seeds or the plant propagation material.

In an embodiment the enzyme is cutinase, wherein the cutinase of SEQ no. 4 is applied in an amount of from 1 ppm to 1 ppb per weight of the seeds or the plant propagation material.

In an embodiment the enzyme is cutinase, wherein the cutinase of SEQ no. 9 is applied in an amount of from 1 ppm to 1 ppb per weight of the seeds or the plant propagation material.

In an embodiment the enzyme is cutinase, wherein the cutinase of SEQ no. 1 is applied in an amount of from 1 pp hundred to 1 ppm per weight of the seeds or the plant propagation material.

In an embodiment the enzyme is cutinase, wherein the cutinase of SEQ no. 2 is applied in an amount of from 1 pp hundred to 1 ppm per weight of the seeds or the plant propagation material.

In an embodiment the enzyme is cutinase, wherein the cutinase of SEQ no. 4 is applied in an amount of from 1 pp hundred to 1 ppm per weight of the seeds or the plant propagation material.

In an embodiment the enzyme is cutinase, wherein the cutinase of SEQ no. 9 is applied in an amount of from 1 pp hundred to 1 ppm per weight of the seeds or the plant propagation material.

In a further embodiment the presently claimed invention is directed to kit of parts comprising, as separate components, at least one active compound for a combined use with at least one enzyme selected from the group consisting of protease, peptidase, cellulase, hemicellulase, pectinase, chitinase, chitosanase, lipase, cutinase esterase and xylanase.

In a further embodiment the presently claimed invention is directed to kit of parts comprising, as separate components, at least one active compound for a combined use with cutinase, wherein the cutinase is as per SEQ NO. 1 or SEQ NO. 2, SEQ NO. 4, SEQ NO. 9.

In an embodiment of the present invention, the method or use or composition or seeds or kit of parts according to any of the preceding embodiments, wherein the at least one active compound is (3R)-3-(2-chlorothiazol-5-yl)-8-methyl-5-oxo-6-phenyl-2,3-dihydrothiazolo[3,2-a]pyrimidin-8-ium-7-olate and the at least one enzyme is cutinase.

In an embodiment of the present invention, the method or use or composition or seeds or kit of parts according to any of the preceding embodiments, wherein the at least one active compound is Fipronil and the at least one enzyme is cutinase.

In an embodiment of the present invention, the method or use or composition or seeds or kit of parts according to any of the preceding embodiments, wherein the at least one active compound is Dinotefuran and the at least one enzyme is cutinase.

In an embodiment of the present invention, the method or use or composition or seeds or kit of parts according to any of the preceding embodiments, wherein the at least one active compound is broflanilide, and the at least one enzyme is cutinase.

In an embodiment of the present invention, the method or use or composition or seeds or kit of parts according to any of the preceding embodiments, wherein the at least one active compound is dimpropyridaz, and the at least one enzyme is cutinase.

In an embodiment of the present invention, the method or use or composition or seeds or kit of parts according to any of the preceding embodiments, wherein the at least one active compound is Clothianidin, and the at least one enzyme is cutinase.

In an embodiment of the present invention, the method or use or composition or seeds or kit of parts according to any of the preceding embodiments, wherein the at least one active compound is Imidacloprid, and the at least one enzyme is cutinase.

In an embodiment of the present invention, the method or use or composition or seeds or kit of parts according to any of the preceding embodiments, wherein the at least one active compound is thiodicarb, and the at least one enzyme is cutinase.

In an embodiment of the present invention, the method of protecting plants or plant propagation material, from attack or infestation by insects, fungi, arachnids or nematodes, protection against weeds, comprising application of:

at least one active compound, and

at least one enzyme which is cutinase

directly and/or indirectly to the plant and/or to plant propagation material by drenching the soil, by drip application onto the soil, by soil injection, by dipping, in-furrow application or by treatment of plant propagation material.

In an embodiment of the present invention, the method of protecting plants or plant propagation material, from attack or infestation by insects, fungi, arachnids or nematodes, protection against weeds, comprising application of:

at least one active compound, and

at least one enzyme which is cutinase

directly and/or indirectly to plant propagation material which is the seed

by drenching the soil, by drip application onto the soil, by soil injection, by dipping, in-furrow application or by treatment of plant propagation material.

In an embodiment of the present invention, the method of protecting plants or plant propagation material, from attack or infestation by insects, fungi, arachnids or nematodes, protection against weeds, comprising application of:

at least one active compound, and

at least one enzyme which is cutinase, wherein the enzyme cutinase comprises an amino acid sequence having 85% identity to SEQ ID NO: 1, SEQ ID NO: 2, SEQ ID NO: 4 or SEQ ID NO: 9,

directly and/or indirectly to plant propagation material which is the seed

by drenching the soil, by drip application onto the soil, by soil injection, by dipping, in-furrow application or by treatment of plant propagation material.

In an embodiment of the present invention, the use of at least one active compound and at least one enzyme which is cutinase for protecting plants or plant propagation material from attack or infestation by insects, fungi, arachnids or nematodes or protection against weeds, wherein the at least one active compound and the at least one enzyme which is cutinase is applied directly and/or indirectly to the plant and/or to plant propagation material by drenching the soil, by drip application onto the soil, by soil injection, by dipping, in-furrow application or by treatment of plant propagation material.

In an embodiment of the present invention, the use of at least one active compound and at least one enzyme which is cutinase for protecting plant propagation material from attack or infestation by insects, fungi, arachnids or nematodes or protection against weeds, wherein the at least one active compound and the at least one enzyme which is cutinase is applied directly and/or indirectly to the plant propagation material which is the seed by drenching the soil, by drip application onto the soil, by soil injection, by dipping, in-furrow application or by treatment of plant propagation material.

In an embodiment of the present invention, the use of at least one active compound and at least one enzyme which is cutinase for protecting plant propagation material from attack or infestation by insects, fungi, arachnids or nematodes or protection against weeds, wherein the at least one active compound and the at least one enzyme which is cutinase, wherein the enzyme cutinase comprises an amino acid sequence having 85% identity to SEQ ID NO: 1, SEQ ID NO: 2, SEQ ID NO: 4 or SEQ ID NO: 9, is applied directly and/or indirectly to the plant propagation material, which is the seed by drenching the soil, by drip application onto the soil, by soil injection, by dipping, in-furrow application or by treatment of plant propagation material.

### Embodiments

In the following, there is provided a list of embodiments to further illustrate the present disclosure without intending to limit the disclosure to the specific embodiments listed below.
1. Method of protecting plants or plant propagation material, from attack or infestation by insects, fungi, arachnids or nematodes, protection against weeds, comprising application of:
   at least one active compound, and
   at least one enzyme selected from the group consisting of protease, peptidase, cellulase, hemicellulase, pectinase, chitinase, chitosanase, lipase, cutinase esterase and xylanase, directly and/or indirectly to the plant and/or to plant propagation material by drenching the soil, by drip application onto the soil, by soil injection, by dipping, in-furrow application or by treatment of plant propagation material.
2. Use of at least one active compound and at least one enzyme selected from the group consisting of protease, peptidase, cellulase, hemicellulase, pectinase, chitinase, chitosanase, lipase, cutinase, esterase and xylanase for protecting plants or plant propagation material from attack or infestation by insects, fungi, arachnids or nematodes or protection against weeds, wherein the at least one active compound and the at least one enzyme is applied directly and/or indirectly to the plant and/or to plant propagation material by drenching the soil, by drip application onto the soil, by soil injection, by dipping, in-furrow application or by treatment of plant propagation material.
3. The method or use according to embodiment 1 or 2, wherein the plant propagation material is seeds.
4. The method or use according to any of the embodiments 1 to 3, wherein the seeds are of transgenic plant.
5. The method or use according to any of embodiments 3 to 4, wherein the wherein the plant roots and shoots resulting from the treated seeds are protected.
6. The method or use according to any of the embodiments 1 to 5, wherein the at least one active compound is selected from the group consisting of insecticides, fungicides, herbicides acaricides and nematicides.
7. The method or use according to embodiment 6, wherein the insecticides, acaricides and nematicides are selected from the groups M.1 to M.UN. X consisting of:
   M.1 AChE inhibitors: aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb, triazamate; acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion;
   M.2. GABA-gated chloride channel antagonists: cyclodiene organochlorine compounds: endosulfan, chlordane; phenylpyrazoles: ethiprole, fipronil, flufiprole, pyrafluprole, pyriprole;
   M.3 Sodium channel modulators: pyrethroids: acrinathrin, allethrin, d-cis-trans allethrin, d-trans allethrin, bifenthrin, kappa-bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, taufluvalinate, halfenprox, heptafluthrin, imiprothrin, meperfluthrin, metofluthrin, momfluorothrin, epsilon-momfluorothrin, permethrin, phenothrin, prallethrin, profluthrin, pyrethrin (pyrethrum), resmethrin, silafluofen, tefluthrin, kappa-tefluthrin, tetramethylfluthrin, tetramethrin, tralomethrin, transfluthrin; sodium channel modulatorse.g.: DDT, methoxychlor;
   M.4 nAChR agonists: neonicotinoids: acetamiprid, clothianidin, cycloxaprid, dinotefuran, imidacloprid, nitenpyram, thiacloprid, thiamethoxam; 4,5-Dihydro-N-nitro-1-(2-oxiranylmethyl)-1H-imidazol-2-amine, (2E-)-1-[(6-Chloropyridin-3-yl)methyl]-N′-nitro-2-pentylidenehydrazinecarboximidamide; 1-[(6-Chloropyridin-3-yl)methyl]-7-methyl-8-nitro-5-propoxy-1,2,3,5,6,7-hexahydroimidazo[1,2-a]pyridine; nicotine; sulfoxaflor; flupyradifurone; triflumezopyrim, (3R)-3-(2-chlorothiazol-5-yl)-8-methyl-5-oxo-6-phenyl-2,3-dihydrothiazolo[3,2-a]pyrimidin-8-ium-7-olate, (3S)-3-(6-chloro-3-pyridyl)-8-methyl-5-oxo-6-phenyl-2,3-dihydrothiazolo[3,2-a]pyrimidin-8-ium-7-olate, (3S)-8-methyl-5-oxo-6-phenyl-3-pyrimidin-5-yl-2,3-dihydrothiazolo[3,2-a]pyrimidin-8-ium-7-olate, (3R)-3-(2-chlorothiazol-5-yl)-8-methyl-5-oxo-6-[3-(trifluoromethyl)phenyl]-2,3-dihydrothiazolo[3,2-a]pyrimidin-8-ium-7-olate; (3R)-3-(2-chlorothiazol-5-yl)-6-(3,5-dichlorophenyl)-8-methyl-5-oxo-2,3-dihydrothiazolo[3,2-a]pyrimidin-8-ium-7-olate, (3R)-3-(2-chlorothiazol-5-yl)-8-ethyl-5-oxo-6-phenyl-2,3-dihydrothiazolo[3,2-a]pyrimidin-8-ium-7-olate;
   M.5 Nicotinic acetylcholine receptor allosteric activators: spinosad, spinetoram;
   M.6 Chloride channel activators: abamectin, emamectin benzoate, ivermectin, lepimectin, milbemectin;
   M.7 Juvenile hormone mimics e.g.: hydroprene, kinoprene, methoprene; fenoxycarb, pyriproxyfen;
   M.8 miscellaneous multi-site inhibitors: CH3Br, other alkyl halides, chloropicrin, sulfuryl fluoride, borax, tartar emetic;
   M.9 Chordotonal organ TRPV channel modulators: pymetrozine; pyrifluquinazon;
   M.10 Mite growth inhibitors: clofentezine, hexythiazox, diflovidazin, etoxazole;
   M.11 Microbial disruptors of insect midgut membranes: Bacillus thuringiensis, Bacillus sphaericus, and insecticidal proteins they producee.g.: Bacillus thuringiensis subsp. israelensis, Bacillus sphaericus, Bacillus thuringiensis subsp. aizawai, Bacillus thuringiensis subsp. kurstaki, Bacillus thuringiensis subsp. tenebrionis, Bt crop proteins: Cry1Ab, Cry1Ac, Cry1Fa, Cry2Ab, mCry3A, Cry3Ab, Cry3Bb, Cry34/35Ab1;
   M.12 Inhibitors of mitochondrial ATP synthase: diafenthiuron, organotin miticidese.g.: azocyclotin, cyhexatin, fenbutatin oxide, propargite, tetradifon;
   M.13 Uncouplers of oxidative phosphorylation via disruption of the proton gradient: chlorfenapyr, DNOC, sulfluramid;
   M.14 nAChR channel blockers: nereistoxin analogues bensultap, cartap hydrochloride, thiocyclam, thiosultap-sodium;
   M.15 Inhibitors of the chitin biosynthesis type e.g.: bistrifluron, chlorfluazuron, diflubenzuron, flucycloxuron, flufenoxuron, hexaflumuron, lufenuron, novaluron, noviflumuron, teflubenzuron, triflumuron;
   M.16 Inhibitors of the chitin biosynthesis type 1: buprofezin;
   M.17 Moulting disruptors: Dipteran, cyromazine;
   M.18 Ecdyson receptor agonistse.g.: methoxyfenozide, tebufenozide, halofenozide, fufenozide, chromafenozide;
   M.19 Octopamin receptor agonists: amitraz;
   M.20 Mitochondrial complex III electron transport inhibitors: hydramethylnon, acequinocyl, fluacrypyrim; bifenazate;
   M.21 METI acaricides and insecticides e.g.: fenazaquin, fenpyroximate, pyrimidifen, pyridaben, tebufenpyrad, tolfenpyrad, rotenone;
   M.22 Voltage-dependent sodium channel blockers: indoxacarb, metaflumizone, 2-[2-(4-cyanophenyl)-1-[3-(trifluoromethyl)phenyl]¬ethylidene]-N-[4-(difluoromethoxy)phenyl]-hydrazinecarboxamide, N-(3-chloro-2-methyl¬phenyl)-2-[(4-chlorophenyl)[4-[methyl(methylsulfonyl)amino]phenyl]¬methylene]-hydrazinecarboxamide;
   M.23 Inhibitors of the of acetyl CoA carboxylase e.g.: spirodiclofen, spiromesifen, spirotetramat; spiropidion;
   M.24 Mitochondrial complex IV electron transport inhibitors: e.g. aluminium phosphide, calcium phosphide, zinc phosphide, cyanide;
   M.25 Mitochondrial complex II electron transport inhibitors e.g.: cyenopyrafen, cyflumetofen;
   M.28 Ryanodine receptor-modulators: flubendiamide, chlorantraniliprole, cyantraniliprole, tetraniliprole, (R)-3-chloro-N1-{2-methyl-4-[1,2,2,2-tetrafluoro-1-(trifluoromethyl)ethyl]phenyl}-N2-(1-methyl-2-methylsulfonylethyl)phthalamid, (S)-3-chloro-N1-{2-methyl-4-[1,2,2,2-tetrafluoro-1-(trifluoromethyl)ethyl]phenyl}-N2-(1-methyl-2-methylsulfonylethyl)phthalamide, cyclaniliprole, methyl-2-[3,5-dibromo-2-({[3-bromo-1-(3-chlorpyridin-2-yl)-1H-pyrazol-5-yl]carbonyl}amino)benzoyl]-1,2-dimethylhydrazine-carboxylate; N-[2-(5-amino-1,3,4-thiadiazol-2-yl)-4-chloro-6-methylphenyl]-3-bromo-1-(3-chloro-2-pyridinyl)-1H-pyrazole-5-carboxamide; 3-chloro-1-(3-chloro-2-pyridinyl)-N-[2,4-dichloro-6-[[(1-cyano-1-methylethyl)amino]carbonyl]phenyl]-1H-pyrazole-5-carboxamide; tetrachlorantraniliprole; N-[4-chloro-2-[[(1,1-dimethylethyl)amino]carbonyl]-6-methyl¬phenyl]-1-(3-chloro-2-pyridinyl)-3-(fluoromethoxy)-1H-pyrazole-5-carboxamide; cyhalodiamide;
   M.29: Chordotonal organ Modulators: flonicamid;
   M.UN. Unknown mode of action: afidopyropen, afoxolaner, azadirachtin, amidoflumet, benzoximate, broflanilide, bromopropylate, chinomethionat, cryolite, dicloromezotiaz, dicofol, dimpropyridaz, flufenerim, flometoquin, fluensulfone, fluhexafon, fluopyram, fluralaner, metaldehyde, metoxadiazone, piperonyl butoxide, pyflubumide, pyridalyl, tioxazafen, 11-(4-chloro-2,6-dimethylphenyl)-12-hydroxy-1,4-dioxa-9-azadispiro[4.2.4.2]-tetradec-11-en-10-one, 3-(4′-fluoro-2,4-dimethylbiphenyl-3-yl)-4-hydroxy-8-oxa-1-azaspiro[4.5]dec-3-en-2-one, 1-[2-fluoro-4-methyl-5-[(2,2,2-trifluoroethyl)sulfinyl]phenyl]-3-(trifluoromethyl)-1H-1,2,4-triazole-5-amine, actives on basis of Bacillus firmus (Votivo, I-1582); flupyrimin; fluazaindolizine; 4-[5-(3,5-dichlorophenyl)-5-(trifluoromethyl)-4H-isoxazol-3-yl]-2-methyl-N-(1-oxothietan-3-yl)benzamide; fluxametamide; 5-[3-[2,6-dichloro-4-(3,3-dichloroallyloxy)phenoxy]propoxy]-1H-pyrazole; 4-cyano-N-[2-cyano-5-[[2,6-dibromo-4-[1,2,2,3,3,3-hexafluoro-1-(trifluoromethyl)-propyl]phenyl]carbamoyl]phenyl]-2-methyl-benzamide; 4-cyano-3-[(4-cyano-2-methyl-benzoyl)amino]-N-[2,6-dichloro-4-[1,2,2,3,3,3-hexafluoro-1-(trifluoromethyl)-propyl]phenyl]-2-fluoro-benzamide; N-[5-[[2-chloro-6-cyano-4-[1,2,2,3,3,3-hexafluoro-1-(trifluoromethyl)propyl]phenyl]carbamoyl]-2-cyano-phenyl]-4-cyano-2-methyl-benzamide; N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide; N-[5-[[2-bromo-6-chloro-4-[1,2,2,3,3,3-hexafluoro-1-(trifluoromethyl)-propyl]phenyl]carbamoyl]-2-cyano-phenyl]-4-cyano-2-methyl-benzamide; 4-cyano-N-[2-cyano-5-[[2,6-dichloro-4-[1,2,2,3,3,3-hexafluoro-1-(trifluoromethyl)-propyl]phenyl]carbamoyl]phenyl]-2-methyl-benzamide; 4-cyano-N-[2-cyano-5-[[2,6-dichloro-4-[1,2,2,2-tetrafluoro-1-(trifluoromethyl)ethyl]phenyl]carbamoyl]¬phenyl]-2-methyl-benzamide; N-[5-[[2-bromo-6-chloro-4-[1,2,2,2-tetrafluoro-1-(trifluoromethyl)ethyl]phenyl]carba¬moyl]-2-cyano-phenyl]-4-cyano-2-methyl-benzamide; 2-(1,3-dioxan-2-yl)-6-[2-(3-pyridinyl)-5-thiazolyl]-pyridine; 2-[6-[2-(5-fluoro-3-pyridinyl)-5-thiazolyl]-2-pyridinyl]-pyrimidine; 2-[6-[2-(3-pyridinyl)-5-thiazolyl]-2-pyridinyl]-pyrimidine; N-methylsul¬fonyl-6-[2-(3-pyridyl)thiazol-5-yl]pyridine-2-carboxamide; N-methylsulfonyl-6-[2-(3-pyridyl)thiazol-5-yl]pyridine-2-carboxamide; 1-[(6-chloro-3-pyridinyl)methyl]-1,2,3,5,6,7-hexahydro-5-methoxy-7-methyl-8-nitroimidazo[1,2-a]pyridine; 1-[(6-chloropyridin-3-yl)methyl]-7-methyl-8-nitro-1,2,3,5,6,7-hexahydroimidazo[1,2-a]pyridin-5-ol; N-(1-methylethyl)-2-(3-pyridinyl)-2H-indazole-4-carboxamide; N-cyclopropyl-2-(3-pyridinyl)-2H-indazole-4-carboxamide; N-cyclohexyl-2-(3-pyridinyl)-2H-indazole-4-carboxamide; 2-(3-pyridinyl)-N-(2,2,2-trifluoroethyl)-2H-indazole-4-carboxamide; 2-(3-pyridinyl)-N-[(tetrahydro-2-furanyl)methyl]-2H-indazole-5-carboxamide; methyl 2-[[2-(3-pyridinyl)-2H-indazol-5-yl]carbonyl]hydrazinecarboxylate; N-[(2,2-difluorocyclopropyl)methyl]-2-(3-pyridinyl)-2H-indazole-5-carboxamide; N-(2,2-difluoropropyl)-2-(3-pyridinyl)-2H-indazole-5-carboxamide; 2-(3-pyridinyl)-N-(2-pyrimidinylmethyl)-2H-indazole-5-carboxamide; N-[(5-methyl-2-pyrazinyl)methyl]-2-(3-pyridinyl)-2H-indazole-5-carboxamide, tyclopyrazoflor; sarolaner, lotilaner; N-[4-chloro-3-[[(phenylmethyl)amino]carbonyl]phenyl]-1-methyl-3-(1,1,2,2,2-pentafluoroethyl)-4-(trifluoromethyl)-1H-pyrazole-5-carboxamide; 2-(3-ethylsulfonyl-2-pyridyl)-3-methyl-6-(tri¬fluoromethyl)imidazo[4,5-b]pyridine, 2-[3-ethylsulfonyl-5-(trifluoromethyl)-2-pyridyl]-3-methyl-6-(trifluoromethyl)imidazo[4,5-b]pyridine; Isocycloseram; N-[4-chloro-3-(cyclopropylcarbamo¬yl)phenyl]-2-methyl-5-(1,1,2,2,2-pentafluoroethyl)-4-(trifluoromethyl)pyrazole-3-carboxamide, N-[4-chloro-3-[(1-cyanocyclopropyl)carbamoyl]phenyl]-2-methyl-5-(1,1,2,2,2-pentafluoroethyl)-4-(trifluoromethyl)pyrazole-3-carboxamide; acynonapyr; benzpyrimoxan; tigolaner; oxazosulfyl; [(2S,3R,4R,5S,6S)-3,5-dimethoxy-6-methyl-4-propoxy-tetrahydropyran-2-yl] N-[4-[1-[4-(trifluoromethoxy)phenyl]-1,2,4-triazol-3-yl]phenyl]carbamate; [(2S,3R,4R,5S,6S)-3,4,5-trimethoxy-6-methyl-tetrahydropyran-2-yl] N-[4-[1-[4-(trifluoromethoxy)phenyl]-1,2,4-triazol-3-yl]phenyl]carbamate; [(2S,3R,4R,5S,6S)-3,5-dimethoxy-6-methyl-4-propoxy-tetrahydropyran-2-yl] N-[4-[1-[4-(1,1,2,2,2-pentafluoroethoxy)phenyl]-1,2,4-triazol-3-yl]phenyl]carbamate; [(2S,3R,4R,5S,6S)-3,4,5-trimethoxy-6-methyl-tetrahydropyran-2-yl] N-[4-[1-[4-(1,1,2,2,2-pentafluoroethoxy)phenyl]-1,2,4-triazol-3-yl]phenyl]carbamate; (2Z)-3-(2-isopropylphenyl)-2-[(E)-[4-[l-[4-(trifluoromethoxy)phenyl]-1,2,4-triazol-3-yl]phenyl]methylenehydrazono]thiazolidin-4-one, (2Z)-3-(2-isopro¬pyl¬phenyl)-2-[(E)[4-[l-[4-(1,1,2,2,2-pentafluoroethoxy)phenyl]-1,2,4-triazol-3-yl]phenyl]methylenehydrazono]thiazolidin-4-one; 2-(6-chloro-3-ethylsulfonyl-imidazo[1,2-a]pyridin-2-yl)-3-methyl-6-(trifluoromethyl)imidazo[4,5-b]pyridine, 2-(6-bromo-3-ethylsulfonyl-imidazo[1,2-a]pyridin-2-yl)-3-methyl-6-(trifluoromethyl)imidazo[4,5-b]pyridine, 2-(3-ethylsulfonyl-6-iodo-imidazo[1,2-a]pyridin-2-yl)-3-methyl-6-(trifluoromethyl)imidazo[4,5-b]pyridine, 2-[3-ethylsulfonyl-6-(trifluoromethyl)imidazo[1,2-a]pyridin-2-yl]-3-methyl-6-(trifluoromethyl)imidazo[4,5-b]pyridine, 2-(7-chloro-3-ethylsulfonyl-imidazo[1,2-a]pyridin-2-yl)-3-methyl-6-(trifluoromethyl)imidazo[4,5-b]pyridine, 2-(3-ethylsulfonyl-7-iodo-imidazo[1,2-a]pyridin-2-yl)-3-methyl-6-(trifluoromethyl)imidazo[4,5-b]pyridine, 3-ethylsulfonyl-6-iodo-2-[3-methyl-6-(trifluoromethyl)imidazo[4,5-b]pyridin-2-yl]imidazo[1,2-a]pyridine-8-carbonitrile, 2-[3-ethylsulfonyl-8-fluoro-6-(trifluoromethyl)imidazo[1,2-a]pyridin-2-yl]-3-methyl-6-(trifluoromethyl)imidazo[4,5-b]pyridine, 2-[3-ethylsulfonyl-7-(trifluoromethyl)imidazo[1,2-a]pyridin-2-yl]-3-methyl-6-(trifluoromethylsulfinyl)imidazo[4,5-b]pyridine, 2-[3-ethylsulfonyl-7-(trifluoromethyl)imidazo[1,2-a]pyridin-2-yl]-3-methyl-6-(trifluoromethyl)imidazo[4,5-c]pyridine, 2-(6-bromo-3-ethylsulfonyl-imidazo[1,2-a]pyridin-2-yl)-6-(trifluoromethyl)pyrazolo[4,3-c]pyridine.
8. The method or use according to embodiment 6, wherein the fungicides are selected from the groups F.I to F.XII:
   A) Respiration inhibitors
   Inhibitors of complex III at Qo site: azoxystrobin, coumethoxystrobin, coumoxystrobin, dimoxystrobin, enestroburin, fenaminstrobin, fenoxystrobin/flufenoxystrobin, fluoxastrobin, kresoxim-methyl, mandestrobin, metominostrobin, orysastrobin, picoxystrobin, pyroclostrobin, pyrametostrobin, pyraoxystrobin, trifloxystrobin, 2 (2-(3-(2,6-di¬chlorophenyl)-1-methyl-allylideneaminooxymethyl)-phenyl)-2 methoxyimino-N methyl-acetamide, pyri-bencarb, triclopyricarb/chlorodincarb, famoxadone, fenamidone, methyl-N-[2-[(1,4-dimethyl-5 phenyl-pyrazol-3-yl)oxylmethyl]phenyl]-N-methoxycarbamate, metyltetrapole, (Z,2E) 5 [1-(2,4-dichloro¬phenyl)pyrazol-3-yl]-oxy-2-methoxyimino-N,3-dimethyl-pent-3-en¬amide, (Z,2E) 5 [1 (4-chlorophenyl)pyrazol-3-yl]oxy-2-methoxyimino-N,3-dimethyl-pent-3-enamide, pyriminostrobin, bifujunzhi, 2-(ortho-((2,5-dimethylphenyl-oxy-methylen)phenyl)-3-methoxy-acrylic acid methylester; inhibitors of complex III at Qi site: cyazofamid, amisulbrom, [(6S,7R,8R) 8 benzyl-3-[(3-hydroxy-4-methoxy-pyridine-2-carbonyl)amino]-6-methyl-4,9-di¬oxo-1,5-dioxonan-7-yl] 2-methylpropanoate, fenpicoxamid, florylpicoxamid;
   inhibitors of complex II: benodanil, benzovindiflupyr, bixafen, boscalid, carboxin, fenfuram, fluopyram, flutolanil, fluxapyroxad, furametpyr, isofetamid, isopyrazam, mepronil, oxycarboxin, penflufen, penthiopyrad), pydiflumetofen, pyraziflumid, sedaxane, tecloftalam, thifluzamide, inpyrfluxam, pyrapropoyne, fluindapyr, N-[2-[2-chloro-4-(trifluoro-methyl)phenoxy]phenyl]-3-(difluoromethyl)-5-fluoro-1-methyl-pyrazole-4-carboxamide, methyl (E)-2-[2-[(5-cyano-2-methyl-phenoxy)methyl]phenyl]-3-methoxyprop-2 enoate, isoflucypram, 2-(difluoromethyl)-N-(1,1,3-trimethyl-indan-4 yl)pyridine-3-carboxamide, 2-(difluoromethyl)-N-[(3R)-1,1,3-trimethylindan-4-yl]pyridine-3-carboxamide, 2-(difluoromethyl)-N-(3-ethyl-1,1-dimethyl-indan-4-yl)pyridine-3-carboxamide, 2-(difluoromethyl)-N-[(3R)-3-ethyl-1,1-dimethyl-indan-4-yl]pyridine-3-carboxamide, 2-(difluoromethyl)-N-(1,1-dimethyl-3-propyl-indan-4-yl)pyridine-3-carboxamide, 2-(difluoromethyl)-N-[(3R)-1,1-dimethyl-3-propyl-indan-4-yl]pyridine-3-carboxamide, 2-(difluoromethyl)-N-(3-isobutyl-1,1-dimethyl-indan-4-yl)pyridine-3-carboxamide, 2-(difluoromethyl)-N-[(3R)-3-isobutyl-1,1-dimethyl-indan-4 yl]pyridine-3-carboxamide;
   other respiration inhibitors: diflumetorim; nitrophenyl derivates: binapacryl, dinobuton, dinocap, fluazinam, meptyldinocap, ferimzone; organometal compounds: fentin salts, e.g. fentin-acetate, fentin chloride, fentin hydroxide; ametoctradin; silthiofam;
   B) Sterol biosynthesis inhibitors (SBI fungicides)
   C14 demethylase inhibitors: triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 2 (2,4-di¬fluorophenyl)-1,1-difluoro-3-(tetrazol-1-yl)-1-[5-[4-(2,2,2-trifluoroethoxy)phenyl]-2 pyridyl]propan-2-ol, 2-(2,4-difluorophenyl)-1,1-difluoro-3-(tetrazol-1-yl)-1 [5 [4 (trifluoromethoxy)phenyl]-2-pyridyl]propan-2-ol, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile, ipfentrifluconazole, mefentrifluconazole, 2-(chloromethyl)-2-methyl-5-(p-tolylmethyl)-1 (1,2,4-triazol-1 ylmethyl)cyclopentanol; imidazoles: imazalil, pefurazoate, prochloraz, triflumizol; pyrimidines, pyridines, piperazines: fenarimol, pyrifenox, triforine, [3-(4-chloro-2-fluoro-phenyl)-5-(2,4-di¬fluoro¬phenyl)isoxazol-4-yl]-(3-pyridyl)methanol;
   Delta14-reductase inhibitors: aldimorph, dodemorph, dodemorph-acetate, fenpropimorph, tridemorph, fenpropidin, piperalin, spiroxamine;
   Inhibitors of 3-keto reductase: fenhexamid;
   Other Sterol biosynthesis inhibitors: chlorphenomizole;
   C) Nucleic acid synthesis inhibitors
   phenylamides or acyl amino acid fungicides: benalaxyl, benalaxyl-M, kiralaxyl, metalaxyl, metalaxyl-M, ofurace, oxadixyl;
   other nucleic acid synthesis inhibitors: hymexazole, octhilinone, oxolinic acid, bupirimate, 5-fluorocytosine, 5-fluoro-2-(p-tolylmethoxy)pyrimidin-4 amine, 5-fluoro-2-(4-fluorophenylmethoxy)pyrimidin-4 amine, 5-fluoro-2 (4 chlorophenylmethoxy)pyrimidin-4 amine;
   D) Inhibitors of cell division and cytoskeleton
   tubulin inhibitors: benomyl, carbendazim, fuberidazole, thiabendazole, thiophanate-methyl, pyridachlometyl, N ethyl-2-[(3-ethynyl-8-methyl-6-quinolyl)oxy]butanamide, N-ethyl-2-[(3-ethynyl-8 methyl-6 quinolyl)oxy]-2-methylsulfanyl-acetamide, 2-[(3-ethynyl-8-methyl-6-quinolyl)oxy]-N(2-fluoroethyl)butanamide, 2-[(3-ethynyl-8-methyl-6-quinolyl)oxy]-N-(2-fluoroethyl)-2-methoxy-acetamide, 2-[(3-ethynyl-8-methyl-6-quinolyl)oxy]-N-propyl-butanamide, 2-[(3-ethynyl-8-methyl-6-quinolyl)oxy]-2-methoxy-N-propyl-acetamide, 2-[(3-ethynyl-8-methyl-6-quinolyl)oxy]-2-methylsulfanyl-N-propyl-acetamide, 2 [(3 ethynyl-8-methy-6-quinolyl)oxy]-N-(2-fluoroethyl)-2-methylsulfanyl-acetamide, 4-(2-bromo-4-fluoro-phenyl)-N-(2-chloro-6-fluoro-phenyl)-2,5-dimethyl-pyrazol-3 amine;
   other cell division inhibitors: diethofencarb, ethaboxam, pencycuron, fluopicolide, zoxamide, metrafenone, pyriofenone, phenamacril;
   E) Inhibitors of amino acid and protein synthesis
   methionine synthesis inhibitors: cyprodinil, mepanipyrim, pyrimethanil;
   protein synthesis inhibitors: blasticidin-S, kasugamycin, kasugamycin hydrochloride-hydrate, mildiomycin, streptomycin, oxytetracyclin;
   F) Signal transduction inhibitors
   MAP/histidine kinase inhibitors: fluoroimid, iprodione, procymidone, vinclozolin, fludioxonil;
   G protein inhibitors: quinoxyfen;
   G) Lipid and membrane synthesis inhibitors
   Phospholipid biosynthesis inhibitors: edifenphos, iprobenfos, pyrazophos, isoprothiolane; lipid peroxidation: dicloran, quintozene, tecnazene, tolclofos-methyl, biphenyl, chloroneb, etridiazole, zinc thiazole;
   phospholipid biosynthesis and cell wall deposition: dimethomorph, flumorph, mandipropamid, pyrimorph, benthiavalicarb, iprovalicarb, valifenalate;
   compounds affecting cell membrane permeability and fatty acides: propamocarb;
   inhibitors of oxysterol binding protein: oxathiapiprolin, fluoxapiprolin, 4-[1-[2-[3-(difluoromethyl)-5-methyl-pyrazol-1-yl]acetyl]-4-piperidyl]-N-tetralin-1-yl-pyridine-2-carboxamide, 4-[1-[2-[3,5-bis(difluoromethyl)pyrazol-1-yl]acetyl]-4-piperidyl]-N-tetralin-1-yl-pyridine-2-carboxamide, 4-[1-[2-[3-(difluoromethyl)-5-(trifluoromethyl)pyrazol-1-yl]acetyl]-4-piperidyl]-N-tetralin-1-yl-pyridine-2-carboxamide, 4-[1-[2-[5-cyclopropyl-3-(difluoromethyl)pyrazol-1-yl]acetyl]-4-piperidyl]-N-tetralin-1-yl-pyri¬dine-2-carboxamide, 4-[1-[2-[5-methyl-3-(trifluoromethyl)pyrazol-1-yl]acetyl]-4-piperidyl]-N-tetralin-1-yl-pyridine-2-carboxamide, 4-[1-[2-[5-(difluoromethyl)-3-(trifluoro¬methyl)pyrazol-1-yl]acetyl]-4-piperidyl]-N-tetralin-1-yl-pyridine-2-carboxamide, 4 [1 [2-[3,5-bis(trifluoromethyl)pyrazol-1-yl]acetyl]-4-piperidyl]-N-tetralin-1-yl-pyridine-2-carboxamide, (4-[1-[2-[5-cyclopropyl-3-(trifluoromethyl)pyrazol-1-yl]acetyl]-4-piperidyl]-N-tetralin-1-yl-pyridine-2-carboxamide;
   H) Inhibitors with Multi Site Action
   inorganic active substances: Bordeaux mixture, copper, copper acetate, copper hydroxide, copper oxychloride, basic copper sulfate, sulfur;
   thio- and dithiocarbamates: ferbam, mancozeb, maneb, metam, metiram, propineb, thiram, zineb, ziram;
   organochlorine compounds: anilazine, chlorothalonil, captafol, captan, folpet, dichlofluanid, dichlorophen, hexachlorobenzene, pentachlorphenole and its salts, phthalide, tolylfluanid;
   guanidines and others: guanidine, dodine, dodine free base, guazatine, guazatine-acetate, iminoctadine, iminoctadine-triacetate, iminoctadine-tris(albesilate), dithianon, 2,6-di¬methyl-1H,5H-[1,4]dithiino[2,3-c:5,6-c′]dipyrrole-1,3,5,7(2H,6H)-tetraone;
   I) Cell wall synthesis inhibitors
   inhibitors of glucan synthesis: validamycin, polyoxin B;
   melanin synthesis inhibitors: pyroquilon, tricyclazole, carpropamid, dicyclomet, fenoxanil;
   J) Plant defence inducers
   acibenzolar-S-methyl, probenazole, isotianil, tiadinil, prohexadione-calcium; phosphonates: fosetyl, fosetyl-aluminum, phosphorous acid and its salts, calcium phosphonate, potassium phosphonate, potassium or sodium bicarbonate, 4 cyclopropyl-N-(2,4-dimethoxyphenyl)thiadiazole-5-carboxamide;
   K) Unknown mode of action
   bronopol, chinomethionat, cyflufenamid, cymoxanil, dazomet, debacarb, diclocymet, diclomezine, difenzoquat, difenzoquat-methylsulfate, diphenylamin, fenitropan, fenpyrazamine, flumetover, flusulfamide, flutianil, harpin, methasulfocarb, nitrapyrin, nitrothal-isopropyl, tolprocarb, oxin-copper, proquinazid, tebufloquin, tecloftalam, triazoxide, N′-(4-(4-chloro-3-trifluoromethyl-phenoxy)-2,5-dimethyl-phenyl)-N-ethyl-N methyl formamidine, N′ (4-(4-fluoro-3-trifluoromethyl-phenoxy)-2,5-dimethyl-phenyl)-N-ethyl-N-methyl formamidine, N′-[4-[[3-[(4-chlorophenyl)methyl]-1,2,4-thiadiazol-5-yl]oxy]-2,5-dimethyl-phenyl]-N-ethyl-N-methyl-formamidine, N′-(5-bromo-6-indan-2-yloxy-2-methyl-3-pyridyl)-N-ethyl-N-methyl-formamidine, N′-[5-bromo-6-[1-(3,5-difluorophenyl)ethoxy]-2-methyl-3-pyridyl]-N-ethyl-N-methyl-formamidine, N′-[5-bromo-6-(4-isopropylcyclohexoxy)-2-methyl-3-pyridyl]-N-ethyl-N-methyl-formamidine, N′ [5 bromo-2-methyl-6-(1-phenylethoxy)-3-pyridyl]-N-ethyl-N-methyl-formamidine, N′-(2-methyl-5-trifluoromethyl-4-(3-trimethylsilanyl-prop¬oxy)-phenyl)-N-ethyl-N-methyl formamidine, N′-(5-difluoromethyl-2 methyl-4-(3-trimethylsilanyl-propoxy)-phenyl)-N-ethyl-N-methyl formamidine, 2-(4-chloro-phenyl)-N-[4-(3,4-dimethoxy-phenyl)isoxazol-5 yl]-2-prop-2-ynyloxy-acetamide, 3 [5-(4-chloro-phenyl)-2,3-dimethylisoxazolidin-3-yl]-pyridine (pyrisoxazole) 3-[5-(4-methylphenyl)-2,3-dimethylisoxazolidin-3 yl]-pyridine, 5-chloro-1 (4,6-di¬methoxy-pyrimidin-2-yl)-2-methyl-1H-benzoimidazole, ethyl (Z) 3 amino-2-cyano-3-phenyl-prop-2-enoate (K.1.40), picarbutrazox, pentyl N-[6-[[(Z)-[(1-methyltetrazol-5-yl)-phenyl-methylene]amino]oxymethyl]-2-pyridyl]carbamate, but-3-ynyl N-[6-[[(Z)-[(1-methyltetrazol-5-yl)-phenyl-methylene]amino]oxymethyl]-2-pyridyl]carbamate, ipflufenoquin, quinofumelin, benziothiazolinone, bromothalonil, 2-(6-benzyl-2-pyridyl)quinazoline, 2-[6-(3-fluoro-4 methoxy-phenyl)-5-methyl-2-pyridyl]-quinazoline, dichlobentiazox, N′-(2,5-dimethyl-4-phenoxy-phenyl)-N-ethyl-N-methyl-formamidine, pyrifenamine, fluopimomide, N′-[5-bromo-2-methyl-6-(1-methyl-2 propoxy-ethoxy)-3-pyridyl]-N-ethyl-N-methyl-formamidine;
9. The method or use according to embodiment 6, wherein the herbicides are selected from the group consisting of:
   Acetamides: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor;
   Amino acid derivatives: bilanafos, glyphosate, glufosinate, sulfosate;
   Aryloxyphenoxypropionates: clodinafop, cyhalofop-butyl, fenoxaprop, fluazifop, haloxyfop, metamifop, propaquizafop, quizalofop, quizalofop-P-tefuryl;
   Bipyridyls: diquat, paraquat;
   (thio)carbamates: asulam, butylate, carbetamide, desmedipham, dimepiperate, eptam (EPTC), esprocarb, molinate, orbencarb, phenmedipham, prosulfocarb, pyributicarb, thiobencarb, triallate;
   Cyclohexanediones: butroxydim, clethodim, cycloxydim, profoxydim, sethoxydim, tepraloxydim, tralkoxydim;
   Dinitroanilines: benfluralin, ethalfluralin, oryzalin, pendimethalin, prodiamine, trifluralin;
   Diphenyl ethers: acifluorfen, aclonifen, bifenox, diclofop, ethoxyfen, fomesafen, lactofen, oxyfluorfen;
   Hydroxybenzonitriles: bomoxynil, dichlobenil, ioxynil;
   Imidazolinones: imazamethabenz, imazamox, imazapic, imazapyr, imazaquin, imazethapyr;
   Phenoxy acetic acids: clomeprop, 2,4-dichlorophenoxyacetic acid (2,4-D), 2,4-DB, dichlorprop, MCPA, MCPA-thioethyl, MCPB, Mecoprop;
   Pyrazines: chloridazon, flufenpyr-ethyl, fluthiacet, norflurazon, pyridate;
   Pyridines: aminopyralid, clopyralid, diflufenican, dithiopyr, fluridone, fluroxypyr, picloram, picolinafen, thiazopyr, triclopyr (2-[(3,5,6-trichloro-2-pyridinyl)oxy] acetic acid, butoxyethyl ester);
   Sulfonyl ureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metazosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propylimidazo[1,2-b]pyridazin-3-yl)sulfonyl)-3-(4,6-dimethoxy-pyrimidin-2-yl)urea;
   Triazines: ametryn, atrazine, cyanazine, dimethametryn, ethiozin, hexazinone, metamitron, metribuzin, prometryn, simazine, terbuthylazine, terbutryn, triaziflam; Ureas: chlorotoluron, daimuron, diuron, fluometuron, isoproturon, linuron, methabenzthiazuron, tebuthiuron;
   Other acetolactate synthase inhibitors: bispyribac-sodium, cloransulam-methyl, diclosulam, florasulam, flucarbazone, flumetsulam, metosulam, ortho-sulfamuron, penoxsulam, propoxy carbazone, pyribambenz-propyl, pyribenzoxim, pyriftalid, pyriminobac-methyl, pyrimisulfan, pyrithiobac, pyroxasulfone, pyroxsulam;
   Others: amicarbazone, aminotriazole, anilofos, beflubutamid, benazolin, bencarbazone, benfluresate, benzofenap, bentazone, benzobicyclon, bicyclopyrone, bromacil, bromobutide, butafenacil, butamifos, cafenstrole, carfentrazone, cinidon-ethyl, chlorthal, cinmethylin, clomazone, cumyluron, cyprosulfamide, dicamba, difenzoquat, diflufenzopyr, Drechslera monoceras, endothal, ethofumesate, etobenzanid, fenoxasulfone, fentrazamide, flumiclorac-pentyl, flumioxazin, flupoxam, flurochloridone, flurtamone, indanofan, isoxaben, isoxaflutole, lenacil, propanil, propyzamide, quinclorac, quinmerac, mesotrione, methyl arsenic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, (3-[2-chloro-4-fluoro-5-(3-methyl-2,6-dioxo-4-trifluoromethyl-5 3,6-dihydro-2Hpyrimidin-1-yl)-phenoxy]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methylphenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxyphenyl)-pyridine-2-carboxylic acid methyl ester, and 4-amino-3-chloro-6-(4-chloro-3-dimethylamino-2-fluoro-phenyl)-pyridine-2-carboxylic acid methyl ester.
10. The method or use according to embodiment 1 or 2, wherein the at least one enzyme is cutinase.
11. The method or use according any of the embodiments 1 to 10, wherein the enzyme cutinase comprises an amino acid sequence having 85% identity to SEQ ID NO: 1.
12. The method or use according to any of the embodiments 1 to 10, wherein the enzyme cutinase comprises an amino acid sequence having 85% identity to SEQ ID NO: 2.
13. The method or use according to any of the embodiments 1 to 10, wherein the enzyme cutinase comprises an amino acid sequence having 85% identity to SEQ ID NO: 4.
14. The method or use according to any of the embodiments 1 to 10, wherein the enzyme cutinase comprises an amino acid sequence having 85% identity to SEQ ID NO. 9.
15. The method or use according to any of the embodiments 1 to 14, wherein the at least one active compound and the at least one enzyme is applied by drenching the soil.
16. The method or use according to any of the embodiments 1 to 15, wherein the at least one active compound and the at least one enzyme is applied by drip irrigation.
17. The method or use according to any of the embodiments 1 to 15, wherein the at least one active compound and the at least one enzyme is applied by drip application system.
18. The method or use according to any of the embodiments 1 to 15, wherein the at least one active compound and the at least one enzyme is applied by soil injection.
19. The method or use according to any of the embodiments 1 to 15, wherein the at least one active compound and the at least one enzyme is applied to the plant propagation material.
20. The method or use according to any of the embodiments 1 to 15, wherein the at least one active compound and the at least one enzyme is applied by in-furrow application.
21. The method or use according to any of the embodiments 1 to 20, wherein the at least one active compound is applied in an amount of from 0.1 g to 1 kg per 100 kg of plant propagation material.
22. The method or use according to any of the embodiments 1 to 19, wherein the at least one enzyme is applied in an amount of from 1 ppb to 1 ppt per weight of the plant propagation material.
23. The method or use according to any of embodiments 5 to 22, wherein the plants, the plant propagation material or the plant roots and shoots resulting from the treated plant propagation material are protected from the attack by soil pests or foliar pests.
24. The method or use according to any of the embodiments 3 to 22, wherein the plant or the plant propagation material is selected from the group consisting of row crop seeds and vegetable seeds, spices, herbs, ornamentals, conifers, shrubs, cotton, tropical crops, citrus plants, fruits, nuts and grape vines.
25. The method or use according to embodiment 24, wherein the plant or the plant propagation material is selected from the group consisting of soybean, corn, cotton, rice, wheat, canola and sunflower.
26. A composition for protecting plants or plant propagation material from, attack or infestation by insects, fungi, arachnids or nematodes, protection against weeds comprising: at least one active compound and at least one enzyme selected from the group consisting of protease, peptidase, cellulase, hemicellulase, pectinase, chitinase, chitosanase, lipase, cutinase esterase and xylanase.
27. The composition according to embodiment 26, wherein the at least one active compound is selected from the group consisting of insecticides, fungicides, herbicide, acaricides and nematicides.
28. The composition according to embodiment 26, wherein the at least one enzyme is cutinase.
29. The composition according to embodiment 28, wherein the enzyme cutinase comprises an amino acid sequence having 85% identity to SEQ ID NO:1.
30. The composition according to embodiment 28, wherein the enzyme cutinase comprises an amino acid sequence having 85% identity to SEQ ID NO: 2.
31. The composition according to embodiment 28, wherein the enzyme cutinase comprises an amino acid sequence having 85% identity to SEQ ID NO: 4.
32. The composition according to embodiment 28, wherein the enzyme cutinase comprises an amino acid sequence having 85% identity to SEQ ID NO: 9.
33. The composition according to any of the embodiments 26 to 32, wherein the at least one enzyme is immobilized on a matrix, support or particle.
34. The composition according to any of the embodiments 26 to 33, wherein the composition further comprises at least one auxiliary agent selected from the group consisting of solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.
35. Seeds comprising at least one compound as defined in any of embodiments 1 to 25 and at least one enzyme as defined in any of embodiments 1 to 25 in an amount of from 1 ppb to 1 ppt per weight of the plant propagation material.
36. Kit of parts I comprising, as separate components, at least one active compound as defined in any one of embodiments 1 to 25 for a combined use with at least one enzyme as defined in any of embodiments 1 to 25.
37. The method or use or composition or seeds or kit of parts according to any of the embodiments 1 to 36, wherein the at least one active compound is (3R)-3-(2-chlorothiazol-5-yl)-8-methyl-5-oxo-6-phenyl-2,3-dihydrothiazolo[3,2-a]pyrimidin-8-ium-7-olate and the at least one enzyme is cutinase.

Advantages:
1) The present invention can significantly reduce the use rate of systemic agriculturally active compounds for seed treatment application.
2) The present invention can be potentially used for foliar and in-furrow application to enhance uptake and improve insecticide, fungicide and herbicide efficacy.
3) The present invention has been found to be effective in monocotyledonous and dicotyledonous plants.

### EXAMPLES

The presently claimed invention is illustrated in detail by non-restrictive working examples which follow. More particularly, the test methods specified hereinafter are part of the general disclosure of the application and are not restricted to the specific working examples.

### Example 1

### The Impact of Cutinases on the Uptake of (3R)-3-(2-chlorothiazol-5-yl)-8-methyl-5-oxo-6-phenyl-2,3-dihydrothiazolo[3,2-a]pyrimidin-8-ium-7-olate Uptake in Canola

Materials:

Active ingredient/active compound: (3R)-3-(2-chlorothiazol-5-yl)-8-methyl-5-oxo-6-phenyl-2,3-dihydrothiazolo[3,2-a]pyrimidin-8-ium-7-olate

Enzymes:

CUT-1—Cutinase according to SEQ No. 1

CUT-2—Cutinase according to SEQ No. 2

CUT-4—Cutinase according to SEQ No. 4

CUT-9—Cutinase according to SEQ No. 9

General Seed Treatment (ST) Method:

Canola seeds were treated with formulated product diluted in water by spinning 100 g seed in a 1-liter HEGE bowl while applying treatment slurry. Seeds were spun until dry.

General Method for Coating Efficiency:

To obtain coating efficiency, one hundred treated seeds were randomly sampled, then divided into 10 groups. Each group (10 seeds as a ground) was separately analysed by LC/MS. An average of the Active ingredient (AI) quantity on seeds was assessed.

Plant Incubation and Uptake Analysis Method:

On the day of planting, 24-well plates (10 mL, nonsterile) were filled with a highly organic plug & seedling mix. Water was uniformly applied to the soil surface using a spray nozzle until just before complete saturation of soil column. Soil was tamped down to a level of 1 cm below plate surface. One treated seed was placed on the soil surface of each well and then covered with potting mixture leveled to the plate surface. The top soil surface was sprayed once more to moisten. Plates were then held in an incubator and top-watered as needed with fertilizer water (NPK 16-4-20-3(Ca)-1(Mg)) via spray nozzle. Seven days after planting, both cotyledons from ten plants were sampled for analysis by clipping the point where cotyledon meets stem. Replication was 10×.

Analytical Method:

The seed/excised cotyledons were extracted with acetonitrile solvent (3 mL, containing 1% acetic acid) by homogenization in Teflon tubes using 2010 Geno/Grinder (Spex Sample Prep) for 2 min at 1200 RPM. The homogenate was then mixed with anhydrous sodium sulphate (˜600 mg), sodium acetate (˜100 mg) and vortexed well before centrifugation using Beckman Coulter (Allegra X-14R) centrifuge for 5 min at 3000 RPM. The clear supernatant was then transferred into appropriate vials for LC/MS analysis.

LCMS analyses were performed using Thermo Fisher Scientific Extractive Orbitrap Mass Analyzer with electrospray positive mode of ionization. Thermo Fisher Scientific Surveyor MS Plus Pump and autosampler were used for the chromatographic runs through a column (Sonoma-C18, 2.1×100 mm, 5 μm). The gradient started with 98:2 Water/MeOH (containing 4 mM ammonium formate and 0.1% formic acid) and ending with 98:2 MeOH/Water in about 10 min.

Experimental Results

Experimental data (Table 1) suggested that small batch seed treatment (100 g seeds) yielded variability on seed coating efficiency, implying necessity of coating efficiency analysis for each sample during uptake analysis. For cotyledon analysis, cutinase-1 stood out among 4 cutinases in terms of mean AI uptake (ng/mg leaf) and percent of AI uptake, which was significantly higher than no enzyme control.

TABLE 1
Impact of cutinase on (3R)-3-(2-chlorothiazol-5-yl)-8-methyl-
5-oxo-6-phenyl-2,3-dihydrothiazolo[3,2-a]pyrimidin-8-ium-7-olate
uptake in canola seedlings through seed treatment

| | | | | | |
|---|---|---|---|---|---|
| | No | | | | |
| Canola ST w/ or | Enzyme | | | | |
| w/o cutinase | (Control) | CUT-1 | CUT-2 | CUT-4 | CUT-9 |

Seed Analysis

| | | | | | |
|---|---|---|---|---|---|
| Mean Al (μg/seed) | 20.1 | 15.6 | 18.3 | 19.3 | 18.4 |
| RSD | 12.5 | 13.4 | 13.2 | 8.9 | 14.2 |
| Coating Efficiency | 90.1 | 69.8 | 81.8 | 86.4 | 82.4 |
| (%) | | | | | |

Cotyledon Analysis

| | | | | | |
|---|---|---|---|---|---|
| Mean Al Uptake | 2.29 | 3.04 | 1.87 | 2.16 | 2.22 |
| (ng/mg leaf) | | | | | |
| RSD | 23.2 | 19.1 | 14.6 | 19.6 | 9.7 |
| Al Uptake (%) | 0.40 | 0.60 | 0.39 | 0.43 | 0.43 |

Notes:
1) Values of mean Al uptake (ng/mg leaf) are normalized using mean % Al recovery* (90.9%) and mean % coating efficiency for each treatment group.
2) Al Uptake was calculated based on spiked control, 22340 ng/replicate
3) 1-way ANOVA was used for analysis, the data represented by the same letter do not significantly differ (p =< 0.05).

### Example 2

### Impact of Cutinase on Clothianidin Uptake in Wheat Seedlings by Seed Treatment

Active ingredient/active compound: clothianidin

Enzymes:

CUT-1—Cutinase according to SEQ No. 1

CUT-2—Cutinase according to SEQ No. 2

CUT-4—Cutinase according to SEQ No. 4

Other Material:

RediEarth is a potting mixture—Sunshine®

Experimental Methods

a. Seed treatment: Formulations were diluted and enzymes were dissolved in HPLC water. Solutions were made and mixed immediately before each treatment. For each treatment, 100 g of spring wheat seeds (Triticum sp.) were placed in a 1-L bowl of a HEGE 11 seed treater. Treatment slurry was applied to spinning seeds in an application volume of 1.5 mL including 2.05 mg of lyophilized enzyme-containing cell extracts and seeds were spun until dry. Directly after treatment, seeds were transferred to paper seed pouches and stored in a cooler filed with ice packs until planting.

b. Plant incubation: Before seed treatment, 24-well plates were filled with RediEarth potting mixture sifted through a 3.35-mm mesh sieve. Tap water was uniformly applied to the soil surface using a conical spray nozzle until just before complete saturation of soil column (˜55 mL/plate). The soil was tamped down to a level of 1 cm below plate surface. One hour after treatment, one seed was placed on the soil surface of each well. RediEarth was sifted directly over the wells and the soil line was levelled to the plate surface. The soil surface was moistened again with ˜22 mL water/plate. Plates were then held in a randomized complete block design in an incubator at 25° C., 50% RH containing two FloraSun (F25T8 Plant Growth) lights at 14L:10D. Replication was 10 plates per treatment.

c. Seed coating efficacy: Fifteen treated wheat seeds were grouped and extracted (10× replicates for each treatment rate) with acetonitrile solvent (15 mL) by homogenization. The homogenate was first centrifuged to bring down the solvent/material sticking to the top portion of the tube including the cap using Beckman Coulter (Allegra X-14R) centrifuge for 3 min at 3000 RPM. The collected homogenate was then treated with anhydrous magnesium sulfate (1500 mg), sodium acetate (100 mg) and vortexed well (30s) before further centrifugation for the same duration as above. The supernatant clear solution was then diluted and transferred into appropriate vials for LC/MS analysis.

d. Cotyledon analysis: Fifteen excised pairs of wheat cotyledons were extracted (10× replicates) with acetonitrile solvent (10 mL, containing 1% acetic acid) by homogenization. The homogenate is first centrifuged to bring down the solvent/material sticking to the cap using Beckman Coulter (Allegra X-14R) centrifuge for 3 min at 3000 RPM. The collected homogenate is then treated with anhydrous magnesium sulfate (˜3500 mg), sodium acetate (˜220 mg) and vortexed well (˜30s) before further centrifugation for the same duration as above. The supernatant clear solution is then transferred into appropriate vials for LC/MS analysis without further dilution.

Control samples were prepared by spiking a known amount std clothianidin into 15 pairs of cotyledons (10 replicates) collected from the seedlings of untreated canola seeds and homogenized and processed as above. The recovery of the AI was quantified from the LC/MS data and then used to normalize the results from treated cotyledon sample sets.

LCMS analyses were performed using Thermo Fisher Scientific Q-Exactive Mass Analyzer with electrospray positive mode of ionization. Thermo Fisher Scientific Surveyor MS Plus Pump and autosampler were used for the chromatographic runs through a column (Sonoma-C18, 2.1×100 mm, 5 u). The gradient started with 98:2 Water/MeOH (containing 4 mM ammonium formate and 0.1% formic acid) and ending with 98:2 MeOH/Water in about 10 min.

Experimental Results:

Experimental data (Table 1) indicated that percent clothianidin uptake in wheat seedlings was significantly enhanced by cutinase 1 seed treatment (P=0.002). Although cutinases 2 & 4 also improved the uptake of clothianidin, but improvement was not significant.

TABLE 2
Impact of different cutinases on clothianidin
uptake in wheat by seed treatments

| | | | | Average of | |
|---|---|---|---|---|---|
| | | | | clothianidin | |
| | | Average | | detected | |
| | Theo- | amount | Seed | on 15 | % |
| | retical | detected | coating | pairs of | Clothianidin |
| | loading | on seed | effi- | wheat | uptake in |
| | (ng/15 | (ng/15 | ciency | cotyledons | wheat |
| Samples | seeds) | seeds) | (%) | (ng) | cotyledons* |
| Vector | 4785 | 2433 ± | 50.8 ± | 9.4 ± 2.0 | 0.39 ± 0.08^{a} |
| control | | 156 | 3.3 | | |
| Cutinase | | 2103 ± | 43.9 ± | 12.5 ± 3.2 | 0.59 ± 0.15^{b} |
| 1 | | 160 | 3.3 | | |
| Cutinase | | 2255 ± | 47.1 ± | 11.9 ± 3.4 | 0.53 ± 0.15^{ab} |
| 2 | | 155 | 3.3 | | |
| Cutinase | | 2432 ± | 50.8 ± | 10.8 ± 2.1 | 0.44 ± 0.09^{a} |
| 4 | | 193 | 4.0 | | |

*One way ANOVA test (P = 0.002)

1. Method of protecting plants or plant propagation material, from attack or infestation by insects, fungi, arachnids or nematodes, protection against weeds, comprising application of:
at least one active compound, and
at least one enzyme selected from the group consisting of protease, peptidase, cellulase, hemicellulase, pectinase, chitinase, chitosanase, lipase, cutinase, esterase and xylanase,
directly and/or indirectly to the plant and/or to plant propagation material by drenching the soil, by drip application onto the soil, by soil injection, by dipping, in-furrow application or by treatment of plant propagation material.

2. (canceled)

3. The method according to claim 1, wherein the plant propagation material is seeds.

4. The method according to claim 1, wherein the seeds are of transgenic plant.

5. The method according to claim 3, wherein the plant roots and shoots resulting from the treated seeds are protected.

6. The method according to claim 1, wherein the at least one active compound is selected from the group consisting of insecticides, fungicides, herbicides, acaricides and nematicides.

7. The method according to claim 1, wherein the at least one enzyme is cutinase.

8. The method according to claim 1, wherein the enzyme cutinase comprises an amino acid sequence having 85% identity to SEQ ID NO: 1.

9. The method according to claim 1, wherein the enzyme cutinase comprises an amino acid sequence having 85% identity to SEQ ID NO: 2.

10. The method according to claim 1, wherein the enzyme cutinase comprises an amino acid sequence having 85% identity to SEQ ID NO: 4.

11. The method according to claim 1, wherein the enzyme cutinase comprises an amino acid sequence having 85% identity to SEQ ID NO. 9.

12. The method according to claim 1, wherein the at least one enzyme is applied in an amount of from 1 ppb to 1 ppt per weight of the plant propagation material.

13. The method according to claim 5, wherein the plants, the plant propagation material or the plant roots and shoots resulting from the treated plant propagation material are protected from the attack by soil pests or foliar pests.

14. The method according to claim 3, wherein the plant or the plant propagation material is selected from the group consisting of row crop seeds and vegetable seeds, spices, herbs, ornamentals, conifers, shrubs, cotton, tropical crops, citrus plants, fruits, nuts and grape vines.

15. A composition for protecting plants or plant propagation material from, attack or infestation by insects, fungi, arachnids or nematodes, protection against weeds comprising:
at least one active compound and
at least one enzyme selected from the group consisting of protease, peptidase, cellulase, hemicellulase, pectinase, chitinase, chitosanase, lipase, cutinase, esterase and xylanase.

16. The composition according to claim 15, wherein the at least one active compound is selected from the group consisting of insecticides, fungicides, herbicide, acaricides and nematicides.

17. The composition according to claim 15, wherein the at least one enzyme is cutinase.

18. The composition according to claim 17, wherein the enzyme cutinase comprises an amino acid sequence having 85% identity to SEQ ID NO:1.

19. The composition according to claim 17, wherein the enzyme cutinase comprises an amino acid sequence having 85% identity to SEQ ID NO: 2.

20. The composition according to claim 17, wherein the enzyme cutinase comprises an amino acid sequence having 85% identity to SEQ ID NO: 4.

21. The composition according to claim 17, wherein the enzyme cutinase comprises an amino acid sequence having 85% identity to SEQ ID NO: 9.

22. Seeds comprising at least one active compound and at least one enzyme selected from the group consisting of protease, peptidase, cellulase, hemicellulase, pectinase, chitinase, chitosanase, lipase, cutinase, esterase and xylanase in an amount of from 1 ppb to 1 ppt per weight of the plant propagation material.

23. Kit of parts comprising, as separate components, at least one active compound for a combined use with at least one enzyme selected from the group consisting of protease, peptidase, cellulase, hemicellulase, pectinase, chitinase, chitosanase, lipase, cutinase, esterase and xylanase.

24. The method according to claim 1, wherein the at least one active compound is (3R)-3-(2-chlorothiazol-5-yl)-8-methyl-5-oxo-6-phenyl-2,3-dihydrothiazolo[3,2-a]pyrimidin-8-ium-7-olate and the at least one enzyme is cutinase.


SEQUENCE LISTING (4 sequences)

SEQ ID NO: 1 - protein, 258 aa, Pseudomonas mendocina
  Cutinase 1
APLPDTPGAPFPAVANFDRSGPYTTSSQSEGPSCRIYRPRDLGQGGVRHPVILWGNGTGAGPSTYAGLLSHWASHGFVVAAAETSNAGTGREMLACLDYLVRENDTPYGTYSGKLNTGRVGTSGHSQGGGGSIMAGQDTRVRTTAPIQPYTLGLGHDSASQRRQQGPMFLMSGGGDTIAFPYLNAQPVYRRANVPVFWGERRYVSHFEPVGSGGAYRGPSTAWFRFQLMDDQDARATFYGAQCSLCTSLLWSVERRGL

SEQ ID NO: 2 - protein, 258 aa, Pseudomonas mendocina
  Cutinase 2
APLPDTPGAPFPAVANFDRSGPYTVSSQSEGPSCRIYRPRDLGQGGVRHPVILWGNGTGAGPSTYAGLLSHWASHGFVVAAAETSNAGTGREMLACLDYLVRENDTPYGTYSGKLNTGRVGTSGHSQGGGGSIMAGQDTRVRTTAPIQPYTLGLGHDSASQRRQQGPMFLMSGGGDTIAFPYLNAQPVYRRANVPVFWGERRYVSHFEPVGSGGAYRGPSTAWFRFQLMDDQDARATFYGAQCSLCTSLLWSVERRGL

SEQ ID NO: 3 - protein, 258 aa, Pseudomonas mendocina
  Cutinase 4
APLPDTPGAPLPAVANFDRSGPYATSNQSEGPSCRIYRPSNLGQGGVRHPVILWGNGTGTGPSTYAGLLSHWASHGFVVAAAETSNAGTGREMLACLDYLVRENDNPYGTYAGKLNTGRVGTSGHSQGGGGSIMAGQDTRVRTTAPIQPYTIGLGHDSASQRRQQGPMFLMSGGGDTIAIPYLNAQPVYLRANVPVFWGERRYVSHFEPVGDGGAYRGPSTAWFRFQLMDDQSARGTFYGTLCSLCSSLLWSVERRGF

SEQ ID NO: 4 - protein, 259 aa, Pseudomonas sp. SST3
  Cutinase 9
APLPDTPGAPFPSVSNFDRSGPYATTSRSEGPNCRVYRPATLGQNGVRHPIVLWGNGTGTGPTAYSGLLSHWASHGFVVAAAETSNAGTGEQMLACLDYLVQESNRTYGTYVGVLNTGRVGTSGHSQGGGGSIMAGQDERVSVTAPIQPYTIGLGHDSASQRNQQGPMFLMSGGGDTIAFPYLNAQPVYTRANVPVFWGERRYVSHFEPVGDGGAYRGPSTAWFRYHLMEDETARSTFYGRFCGLCTSVLWTEERKDIE